(19) United States
(12) Patent Application Publication
(10) Pub. No.: US 2026/0020544 A1
(43) Pub. Date: Jan. 22, 2026

(54) METHODS AND SYSTEMS FOR COLLECTING ANNOTATED DATA FOR CREATING A PET HEALTH RISK ASSESSMENT MACHINE MODEL

(71) Applicant: Mars, Incorporated, McLean, VA (US)

(72) Inventors: Nina ROMANOVA, Jersey City, NJ (US); Fernando Rodrigues JUNIOR, São José dos Campos (BR); Katherine BALINGIT, Pasadena, CA (US); Mark PARKINSON, Long Valley, NJ (US); Prateek DHAWALIA, McLean, VA (US)

(21) Appl. No.: 19/275,229

(22) Filed: Jul. 21, 2025

Related U.S. Application Data
(60) Provisional application No. 63/673,970, filed on Jul. 22, 2024.
(60) Provisional application No. 63/720,233, filed on Nov. 14, 2024.
(60) Provisional application No. 63/730,061, filed on Dec. 10, 2024.

(51) Int. Cl. A01K 29/00; G06V 10/70; G06V 40/10

(52) U.S. Cl. CPC A01K 29/007; G06V 10/70; G06V 40/10; G06V 2201/10

(57) ABSTRACT

A computer-implemented method for using an image classifier to identify pet oral conditions and pet dermatological conditions is disclosed. The method includes receiving an indication from a user device to initiate a pet condition analysis process for a pet, collecting pet data corresponding to the pet, outputting a pet image prompt to a user interface of the user device, receiving pet image data via the user interface of the user device, wherein the pet image data includes oral image data of the pet or dermatological image data of the pet, inputting the pet image data and the pet data into a machine-learning model to identify a pet condition and a pet condition recommendation, based on the inputting, receiving the pet condition and the pet condition recommendation from the machine-learning model, and outputting the pet condition and the pet condition recommendation to the user interface of the user device.

### CROSS-REFERENCE TO RELATED APPLICATION(S)

This patent application claims the benefit of priority to U.S. Provisional Application No. 63/673,970, filed on Jul. 22, 2024, U.S. Provisional Application No. 63/720,233, filed on Nov. 14, 2024, and U.S. Provisional Application No. 63/730,061, filed on Dec. 10, 2024, the entireties of which are incorporated herein by reference.

### TECHNICAL FIELD

Various embodiments of this disclosure relate generally to systems and methods for using an image classifier to identify pet oral conditions and/or pet dermatological conditions.

### BACKGROUND

Oral health and dermatological health are critical aspects of overall wellness for pets. However, many pet owners may be inexperienced regarding how to help their pets avoid oral and/or dermatological health issues. For example, many pet owners may be unaware regarding what kind of oral hygiene routine that their pet may need. Dog owners may be unaware that their dogs may need a similar oral hygiene routine to humans (e.g., daily teeth brushing and regular dental check-ups). Such a gap in knowledge may lead to the development and progression of various dental and/or dermatological issues, such as plaque, calculus, gingival abnormalities (e.g., gingivitis and gingival recession), ringworm, hot spots, and the like. Moreover, the dental issues and/or dermatological issues may significantly affect a pet's quality of life. However, diagnosing the dental conditions and/or dermatological conditions may be very difficult for pet owners who do not have specific veterinary training. As a result, many oral health issues and/or dermatological health issues may go unnoticed until the issues may become severe. Thus, a need exists for a pet owner to have the technological capability to detect oral health issues and/or dermatological health issues in the pet owner's pet.

This disclosure is directed to addressing above-referenced challenges. The background description provided herein is for the purpose of generally presenting the context of the disclosure. Unless otherwise indicated herein, the materials described in this section are not admitted to be prior art, or suggestions of the prior art, by inclusion in this section.

### SUMMARY OF THE DISCLOSURE

According to certain aspects of the disclosure, methods and systems are disclosed for using an image classifier to identify pet oral conditions and pet dermatological conditions.

In one aspect, an exemplary embodiment of a computer-implemented method for using an image classifier to identify pet oral conditions and pet dermatological conditions is disclosed. The method may include receiving, by one or more processors, an indication from a user device to initiate a pet condition analysis process for a pet. The method may include, in response to receiving the indication, collecting, by the one or more processors, pet data corresponding to the pet, wherein the pet data includes a breed of the pet, an age of the pet, a weight of the pet, and/or a location of the pet. The method may include, in response to the receiving the pet data, outputting, by the one or more processors, a pet image prompt to a user interface of the user device, wherein the pet image prompt includes a request for image data corresponding to the pet. The method may include, in response to outputting the pet image prompt, receiving, by the one or more processors, pet image data via the user interface of the user device, wherein the pet image data includes oral image data of the pet or dermatological image data of the pet. The method may include inputting, by the one or more processors, the pet image data and the pet data into a machine-learning model to identify a pet condition and a pet condition recommendation. The method may include, based on the inputting, receiving, by the one or more processors, the pet condition and the pet condition recommendation from the machine-learning model, wherein the pet condition corresponds to a pet oral condition or a pet dermatological condition. The method may include outputting, by the one or more processors, the pet condition and the pet condition recommendation to the user interface of the user device.

In a further aspect, an exemplary embodiment of a computer system for using an image classifier to identify pet oral conditions and pet dermatological conditions is disclosed, the computer system comprising at least one memory storing instructions, and at least one processor configured to execute the instructions to perform operations. The operations may include receiving an indication from a user device to initiate a pet condition analysis process for a pet. The operations may include, in response to receiving the indication, collecting pet data corresponding to the pet, wherein the pet data includes a breed of the pet, an age of the pet, a weight of the pet, and/or a location of the pet. The operations may include, in response to the receiving the pet data, outputting a pet image prompt to a user interface of the user device, wherein the pet image prompt includes a request for image data corresponding to the pet. The operations may include, in response to outputting the pet image prompt, receiving pet image data via the user interface of the user device, wherein the pet image data includes oral image data of the pet or dermatological image data of the pet. The operations may include inputting the pet image data and the pet data into a machine-learning model to identify a pet condition and a pet condition recommendation. The operations may include, based on the inputting, receiving the pet condition and the pet condition recommendation from the machine-learning model, wherein the pet condition corresponds to a pet oral condition or a pet dermatological condition. The operations may include outputting the pet condition and the pet condition recommendation to the user interface of the user device.

In a further aspect, an exemplary embodiment of a non-transitory computer-readable medium storing instructions that, when executed by at least one processor, cause the at least one processor to perform operations for using an image classifier to identify pet oral conditions and pet dermatological conditions is disclosed. The operations may include receiving an indication from a user device to initiate a pet condition analysis process for a pet. The operations may include, in response to receiving the indication, collecting pet data corresponding to the pet, wherein the pet data includes a breed of the pet, an age of the pet, a weight of the pet, and/or a location of the pet. The operations may include, in response to the receiving the pet data, outputting a pet image prompt to a user interface of the user device, wherein the pet image prompt includes a request for image data corresponding to the pet. The operations may include, in response to outputting the pet image prompt, receiving pet image data via the user interface of the user device, wherein the pet image data includes oral image data of the pet or dermatological image data of the pet. The operations may include inputting the pet image data and the pet data into a machine-learning model to identify a pet condition and a pet condition recommendation. The operations may include, based on the inputting, receiving the pet condition and the pet condition recommendation from the machine-learning model, wherein the pet condition corresponds to a pet oral condition or a pet dermatological condition. The operations may include outputting the pet condition and the pet condition recommendation to the user interface of the user device.

It is to be understood that both the foregoing general description and the following detailed description are exemplary and explanatory only and are not restrictive of the disclosed embodiments.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary platform environment, according to one or more embodiments.

FIG. 2 depicts an exemplary environment of a pet owner profile and corresponding pet profiles, according to one or more embodiments.

FIG. 3A depicts a flow chart of an exemplary campaign system, according to one or more embodiments.

FIG. 3B depicts a flow chart of an exemplary dermatological detection system, according to one or more embodiments.

FIGS. 4A-Q depict an exemplary mobile application for utilizing an image classifier to collect image data to train one or more machine-learning models, according to one or more embodiments.

FIGS. 5A-R depict an exemplary mobile application for utilizing an image classifier to determine a pet condition and a corresponding recommendation, according to one or more embodiments.

FIG. 6A illustrates a flowchart of an exemplary method of an exemplary embodiment for utilizing an image classifier to identify pet oral conditions and pet dermatological conditions, according to one or more embodiments.

FIG. 6B illustrates a flowchart of an exemplary method of an exemplary embodiment for utilizing an image classifier to determine the image quality of an uploaded image, according to one or more embodiments.

FIG. 6C illustrates a flowchart of an exemplary process for performing an oral image analysis of a dog, according to one or more embodiments.

FIG. 7 depicts an exemplary environment that may be utilized with techniques presented herein, according to one or more embodiments.

FIG. 8 depicts an example of a computing device that may execute the techniques described herein, according to one or more embodiments.

### DETAILED DESCRIPTION OF EMBODIMENTS

According to certain aspects of the disclosure, methods and systems are disclosed for using an image classifier to identify pet oral conditions and pet dermatological conditions. Conventional techniques may not be suitable because conventional techniques may not allow for a real-time analysis and collection of the pet's image data to identify oral and dermatological health conditions. Moreover, conventional techniques may not allow for recommendations regarding how to treat the pet's condition.

A need exists for an integrated hardware, software, and diagnostic solution for analyzing pet image data to identify pet oral conditions and/or pet dermatological conditions. Such a solution may leverage computer vision technology to develop machine-learning models that provide a real-time analysis of pet image data. The machine-learning models may accurately analyze and identify a pet condition based on the pet image data. Additionally, the pet image data and corresponding pet data may be stored for additional training of the machine-learning model. Benefits of the solution include utilizing a machine-learning model to detect subtle patterns in a pet's teeth and gums, where such patterns may be undetectable to the human eye. Additionally, early detection of pet conditions will also reduce the risk of severe health issues and diseases as the pet ages.

As will be discussed in more detail below, in various embodiments, systems and methods are described for using an image classifier to identify pet oral conditions and pet dermatological conditions. The systems and methods may receive, by one or more processors, an indication from a user device to initiate a pet condition analysis process for a pet. The systems and methods may, in response to receiving the indication, collect, by the one or more processors, pet data corresponding to the pet, wherein the pet data includes a breed of the pet, an age of the pet, a weight of the pet, and/or a location of the pet. The systems and methods may, in response to the receiving the pet data, output, by the one or more processors, a pet image prompt to a user interface of the user device, wherein the pet image prompt includes a request for image data corresponding to the pet. The systems and methods may, in response to outputting the pet image prompt, receive, by the one or more processors, pet image data via the user interface of the user device, wherein the pet image data includes oral image data of the pet or dermatological image data of the pet. The systems and methods may input, by the one or more processors, the pet image data and the pet data into a machine-learning model to identify a pet condition and a pet condition recommendation. The systems and methods may, based on the inputting, receive, by the one or more processors, the pet condition and the pet condition recommendation from the machine-learning model, wherein the pet condition corresponds to a pet oral condition or a pet dermatological condition. The systems and methods may output, by the one or more processors, the pet condition and the pet condition recommendation to the user interface of the user device.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of +10% of a stated or understood value.

As used herein, a term such as “user” or the like generally encompasses a future pet owner, future pet owners, pet owner, and/or pet owners. A term such as “pet” or the like generally encompasses a domestic animal, such as a domestic canine, feline, rabbit, ferret, horse, cow, or the like. In exemplary embodiments, “pet” may refer to a canine.

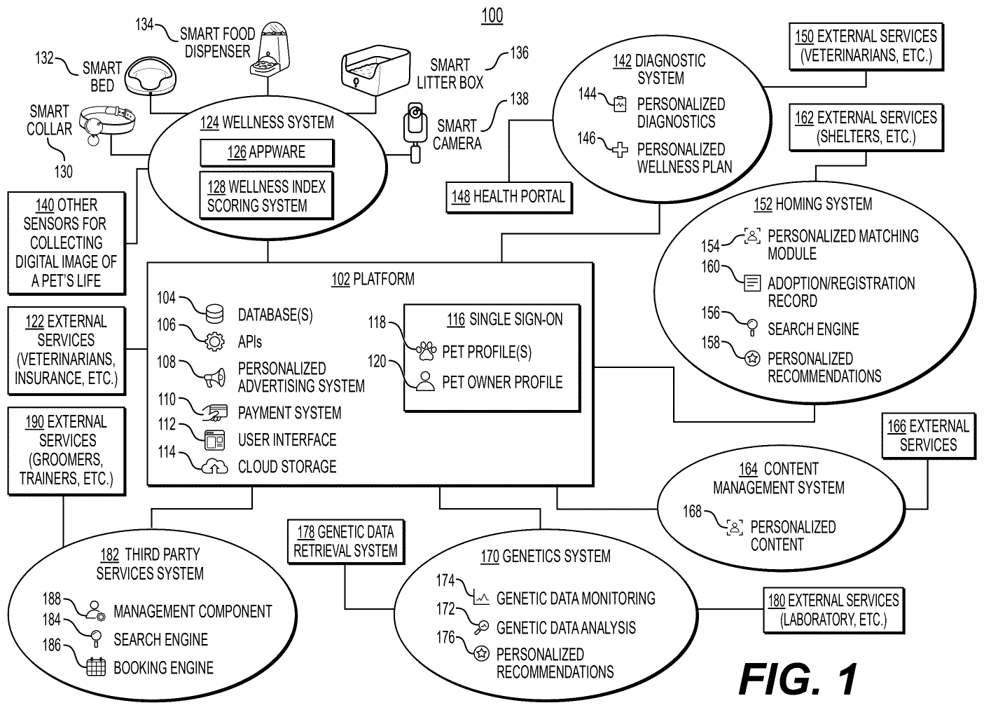

FIG. 1 depicts an exemplary platform environment 100 that may be utilized with the techniques presented herein. More specifically, environment 100 may provide an integrated hardware and software platform for improving pet digitalization by centralizing the pet's information.

Platform 102 may communicate with one or more external systems that may collect, manage, and store different types of pet data and/or pet owner data. Platform 102 may retrieve the pet data and/or pet owner data from the one or more external systems via APIs 106. In some embodiments, platform 102 may store the pet data and/or the pet owner data. For example, platform 102 may store the pet data in pet profile(s) 118. Additionally, for example, platform 102 may store the pet owner data in a pet owner profile 120. The one or more external systems may include at least one of a wellness system 124, a diagnostic system 142, a homing system 152, a content management system 164, a genetics system 170, and/or a third party services system 182. Such external systems are described in more detail below.

Platform 102 may also communicate with one or more external services. In some embodiments, platform 102 may communicate with the one or more external services via APIs 106. External services 122 may include, for example, one or more third party and/or auxiliary systems that integrate and/or communicate with the platform 102 in performing various pet tasks. For example, the external services 122 may include at least one of: a veterinarian, a pet insurance agency, a pet service provider, and the like.

Platform 102 may include database(s) 104 and/or cloud storage 114 that may store information corresponding to one or more pets and/or one or more pet owners. For example, the database(s) 104 and/or cloud storage 114 may store pet profile(s) 118 and/or pet owner profile 120. The database(s) 104 and/or the cloud storage 114 may be located internally or externally to platform 102.

Platform 102 may include a personalized advertising system 108 and/or a payment system 110. The personalized advertising system 108 may create and/or display personalized advertisements to the user. For example, the personalized advertisements may be created based on information contained in pet profile(s) 118 and/or pet owner profile 120. In some embodiments, the personalized advertising system 108 may display the personalized advertisements on a user interface 112 of the platform 102. The payment system 110 may allow the user to create a financial account for a pet and/or perform financial transactions for pet services and/or pet goods (e.g., using pet owner digital wallet 216).

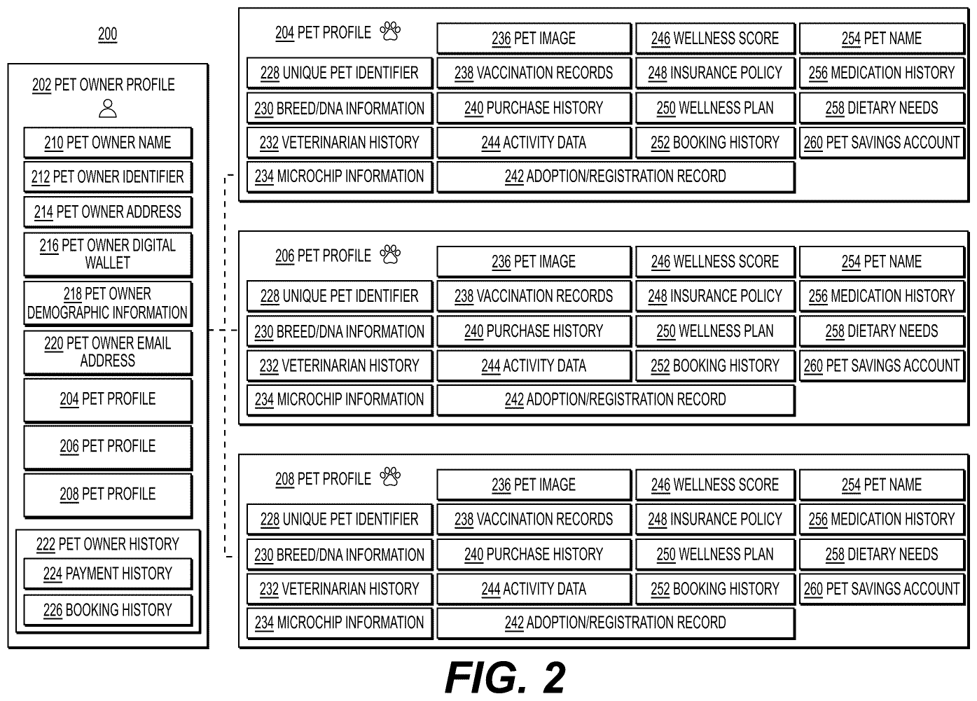

Platform 102 may include a single sign-on 116. The single sign-on 116 may include a unique identifier that may correspond to the pet profile(s) 118 and/or the pet owner profile 120. Each of the pet profile(s) 118 may include information corresponding to a particular pet. The pet owner profile 120 may include information corresponding to a particular pet owner. Additionally, the pet owner profile 120 and/or the pet profile(s) 118 may each have a corresponding avatar and/or virtual presence. The avatar and/or virtual presence may include different attributes that are shared by the pet owner and/or pets. The pet profile(s) 118 and pet owner profile 120 are described in further detail in the description of FIG. 2.

### Wellness System

The wellness system 124 may collect, manage, and/or display wellness data of a pet. The wellness system 124 may be an internal component or an external component of platform 102, where the wellness system 124 may communicate with platform 102 via APIs 106.

The wellness system 124 may collect data (e.g., mobility data) from one or more smart devices. The wellness system 124 may communicate with the one or more smart devices via one or more APIs. Additionally, in some embodiments, the wellness system may use appware 126 to facilitate the communication and/or the management of the one or more smart devices. For example, appware 126 may communicate with one or more smart devices that may run on an external system. Additionally, for example, appware 126 may run on a user device, where the appware 126 provides a user interface to display the data collected by the one or more smart devices. In some embodiments, appware 126 may manage one or more smart devices. The wellness system 124 may communicate with the one or more smart devices by sending one or more requests to the one or more smart devices. The requests may ask the one or more smart devices to send collected wellness data to the wellness system 124. In some embodiments, the one or more smart devices may automatically send wellness data (e.g., mobility data) to the wellness system 124. For example, the one or more smart devices may send the wellness data to the wellness system 124 at regular time intervals (e.g., every 30 seconds, every hour, every day, and the like) and/or whenever new wellness data is collected. In some embodiments, the wellness system 124 may store the wellness data in an internal or external storage. For example, the wellness system 124 may store the wellness data in databases 104 and/or cloud storage 114. Additionally, or alternatively, for example, the wellness system 124 may store the wellness data in the pet profile(s) 118 and/or the pet owner profile 120.

Upon receiving the wellness data from the one or more smart devices, a wellness index scoring system 128 may analyze the wellness data to determine a wellness score. The wellness index scoring system 128 may update the wellness score, where the updating is based on the most recently received wellness data. In some embodiments, the wellness index scoring system 128 may store the wellness score in one or more databases (e.g., database(s) 104) and/or cloud storage (e.g., cloud storage 114). For example, the wellness score may be stored in the pet profile(s) 118 and/or the pet owner profile 120. Additionally, or alternatively, the wellness system 124 may display the wellness score to the user. For example, the wellness system 124 may display the wellness score on a user interface of a user device. This may be accomplished by utilizing the appware 126. Additionally, or alternatively, the wellness system 124 may display the wellness score on one or more of the smart devices.

Example smart devices may include at least one of: a smart collar 130, a smart bed 132, a smart feeder 134, a smart litter box 136, a smart camera 138, and/or the other sensors for collecting a digital image of a pet's life 140.

The smart collar 130 may include a device and/or a sensor that may attach to a pet. For example, the smart collar 130 may attach around the pet's neck The smart collar 130 may detect a pet's activity, location, and eating information, such as physical activity, location, eating habits, drinking habits, and the like. The smart collar 130 may detect and collect the pet's mobility information. The pet's mobility information may be processed to determine one or more metrics, such as the velocity, cadence, and entropy of the pet's gait. For example, the smart collar 130 may detect and collect the speed and direction at which the pet moves at any given point in time. Additionally, for example, the smart collar 130 may detect and collect data related to the inconsistency of the pet's gait (e.g., entropy of the pet's gait). For example, a higher entropy may imply a higher inconsistency detected in the pet's gait, which may be a sign of worse mobility. Conversely, a lower entropy measurement may imply a more consistent gait. The smart collar 130 may collect the activity, location, and eating information of the pet and send such information to the wellness system 124. In some embodiments, the smart collar 130 may automatically send the activity, location, and eating information to the wellness system 124 after a set period of time. In some embodiments, the smart collar 130 may send the activity, location, and eating information in response to a request from the wellness system 124.

The smart bed 132 may include a device and/or a sensor that may be included in a pet bed. The smart bed 132 may track sleeping information corresponding to the pet. The sleeping information may include the amount of time a pet sleeps in the smart bed 132, how frequently the pet gets up from the smart bed 132, if the pet tosses and turns while sleeping, and the like. The smart bed 132 may send such information to the wellness system 124. In some embodiments, the smart bed 132 may automatically send the sleeping information to the wellness system 124 after a set period of time. In some embodiments, the smart bed 132 may send the sleeping information in response to a request from the wellness system 124.

The smart feeder 134 may include a device and/or a sensor that may be included in a pet food feeder. The smart feeder 134 may track how much food is dispensed for the pet to eat. The smart feeder 134 may send such food dispensing information to the wellness system 124. In some embodiments, the smart feeder 134 may automatically send the food dispensing information to the wellness system 124 after a set period of time. In some embodiments, the smart feeder 134 may send the food dispensing information in response to a request from the wellness system 124.

The smart litter box 136 may include a device and/or a sensor that may be included in a litter box. The smart litter box 136 may track a pet's litter box information. The litter box information may include at least one of: how frequently the pet uses the smart litter box 136, what the pet does in the smart litter box 136, and the like. In some embodiments, the smart litter box 136 may automatically send the litter box information to the wellness system 124. In some embodiments, the smart litter box 136 may automatically send the litter box information to the wellness system 124 after a set period of time. In some embodiments, the smart litter box 136 may send the litter box information in response to a request from the wellness system 124.

The smart camera 138 may include a device and/or a sensor that may be included in a camera. The smart camera 138 may capture behavior information of a pet. The pet's behavior information may include physical activity, eating food from the pet's food dish, eating food from a source different from the pet's food dish, drinking from the pet's drinking dish, drinking from a source different from the pet's drinking dish, and the like. In some embodiments, the smart camera 138 may automatically send the behavior information to the wellness system 124 after a set period of time. In some embodiments, the smart camera 138 may send the behavior information in response to a request from the wellness system 124.

The other sensors for collecting a digital image of a pet's life 140 may include one or more devices and/or one or more sensors that collect data for a digital image of the pet's life. Example collected data may include information regarding the pet's eating behavior, sleeping behavior, drinking behavior, playing behavior, and the like. In some embodiments, the other sensors 140 may automatically send the collected data to the wellness system 124 after a set period of time. In some embodiments, the other sensors 140 may send the collected data in response to a request from the wellness system 124.

### Diagnostic System

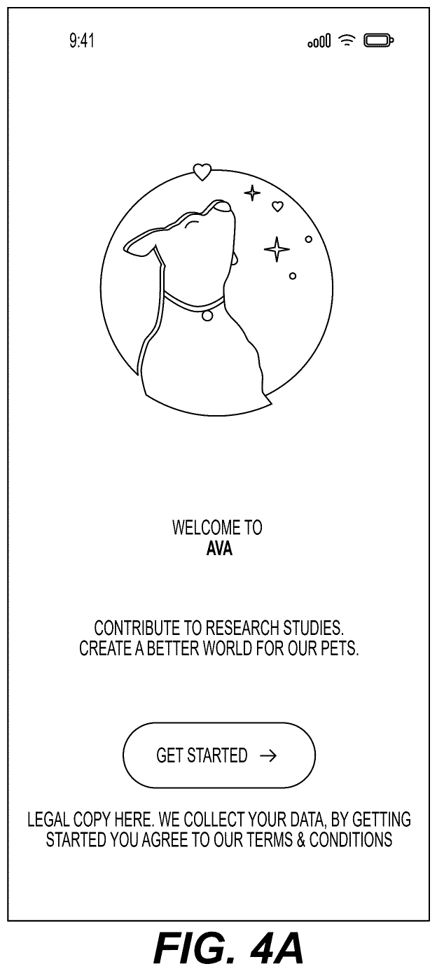

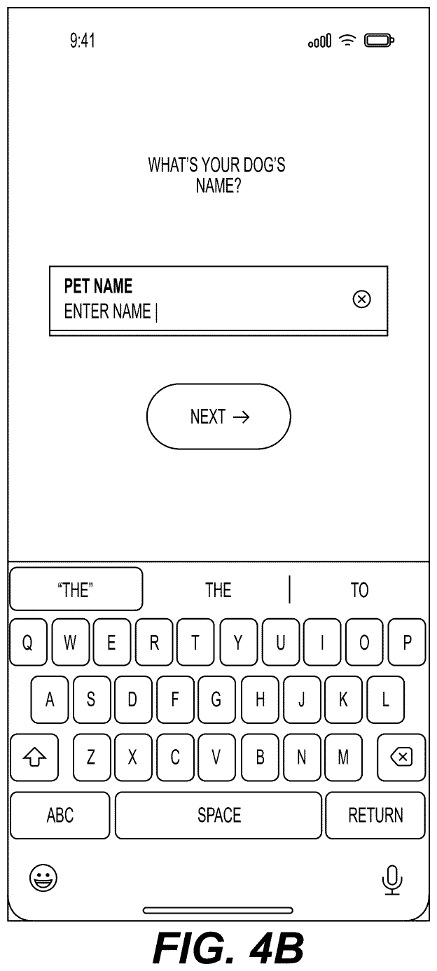

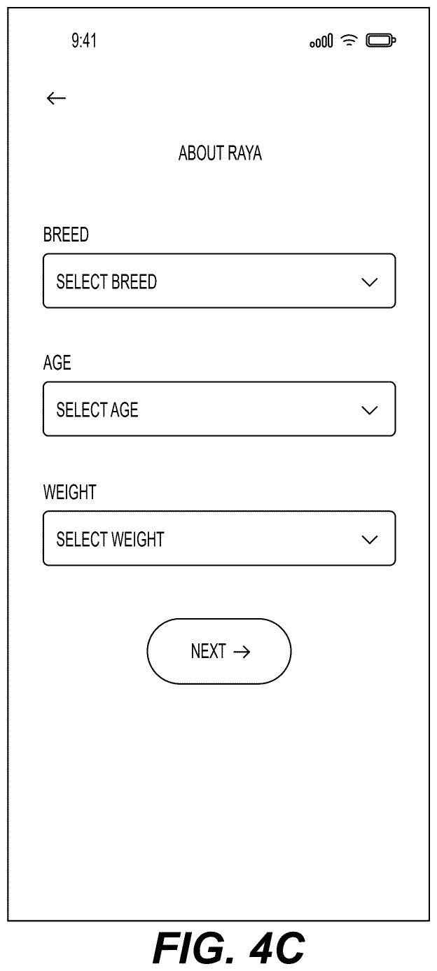

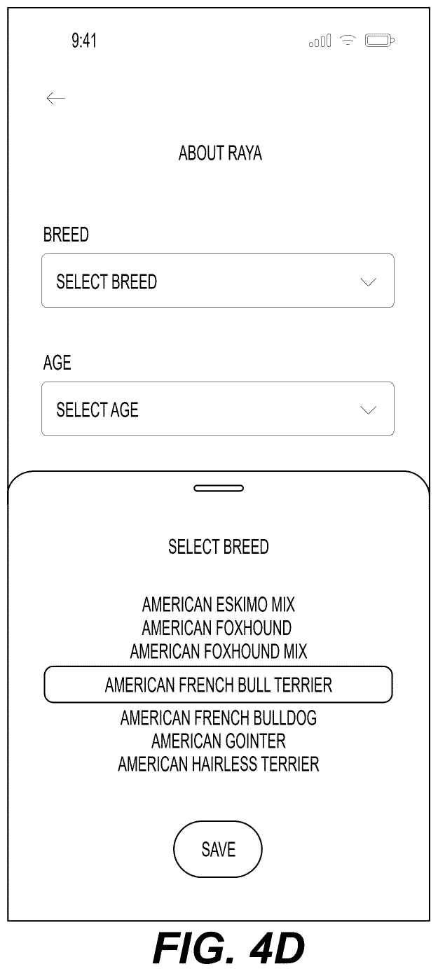

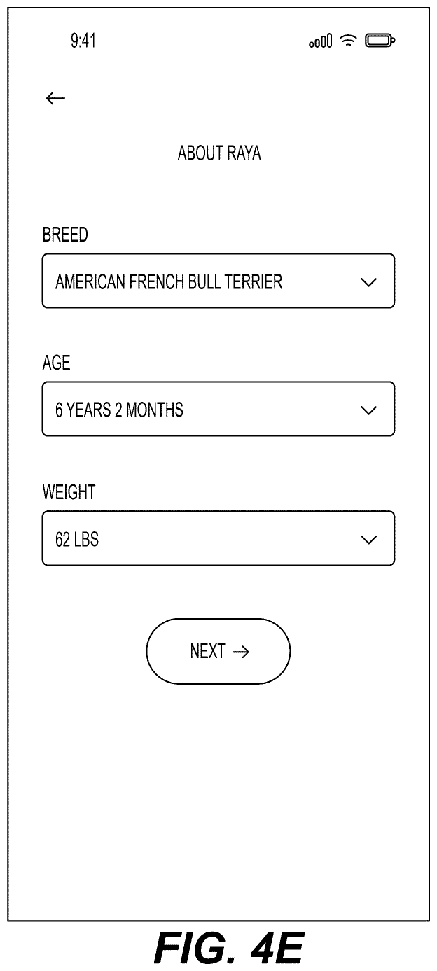

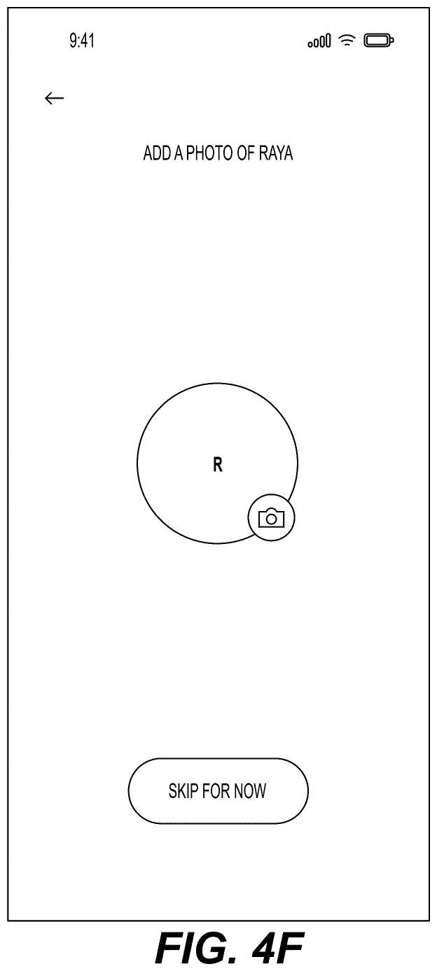

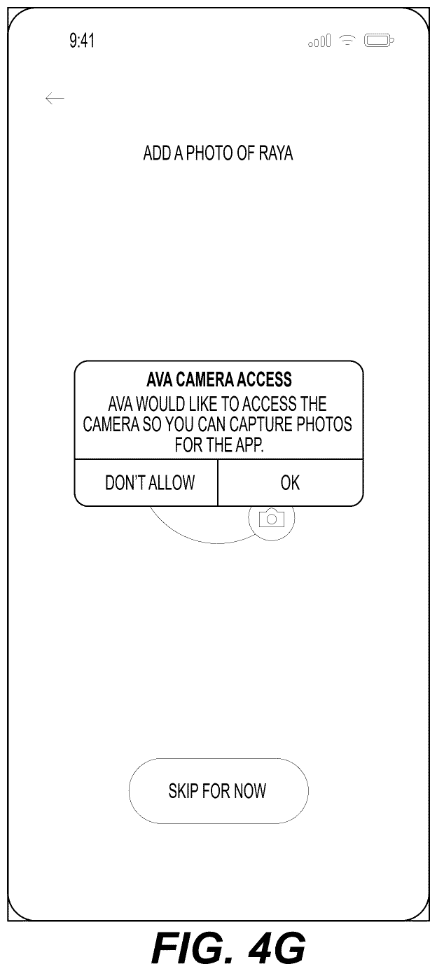

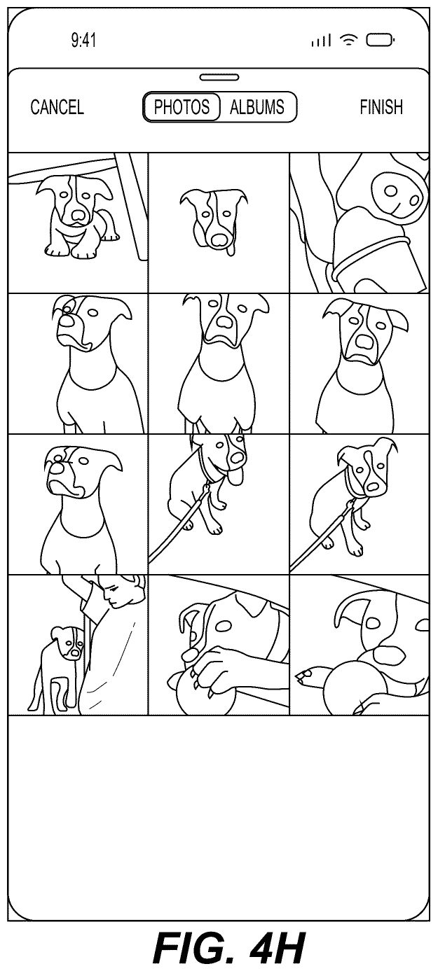

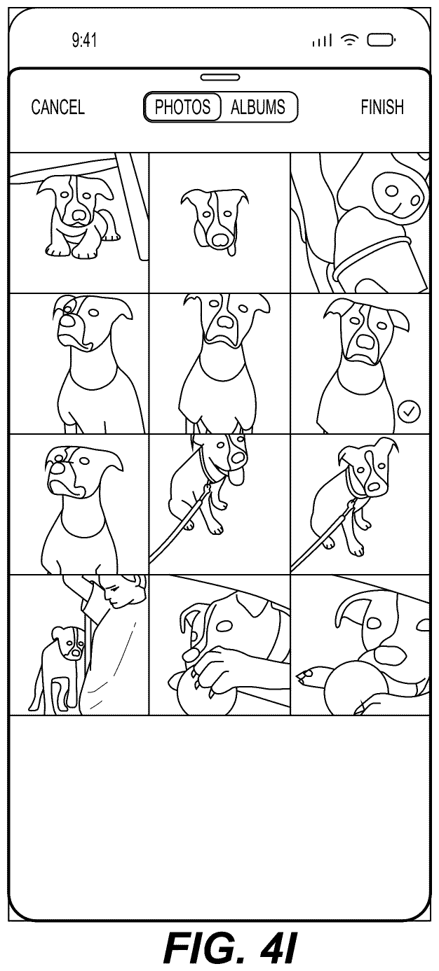

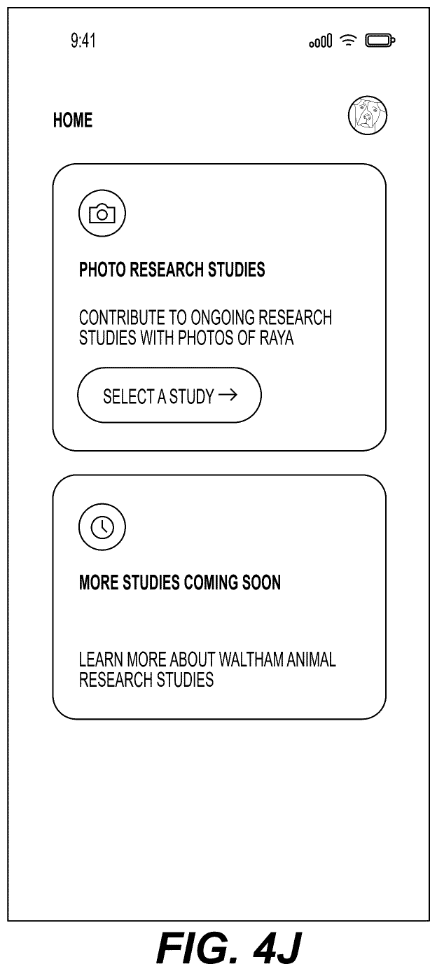

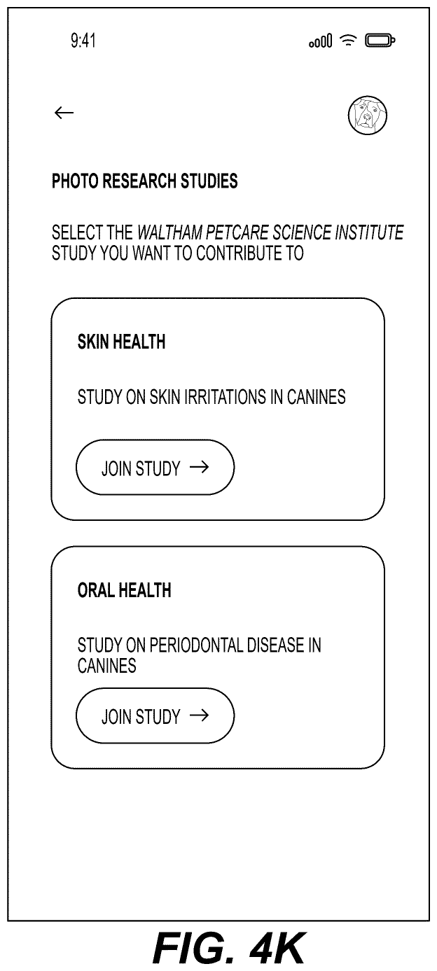

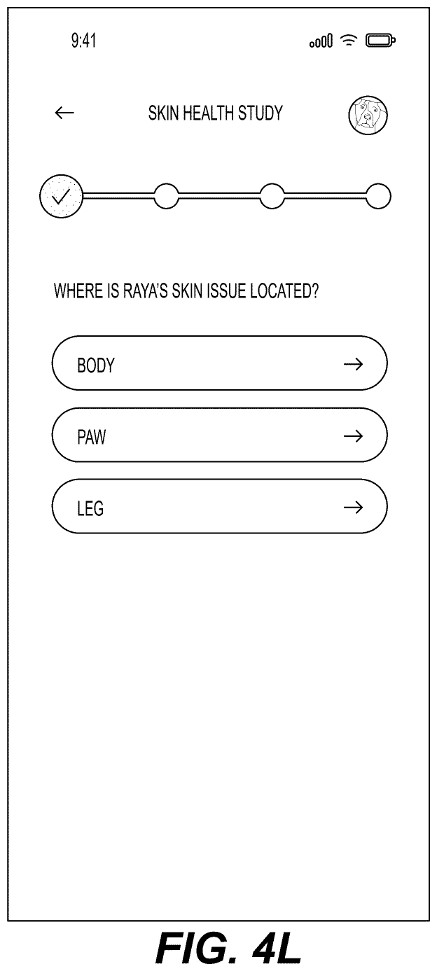

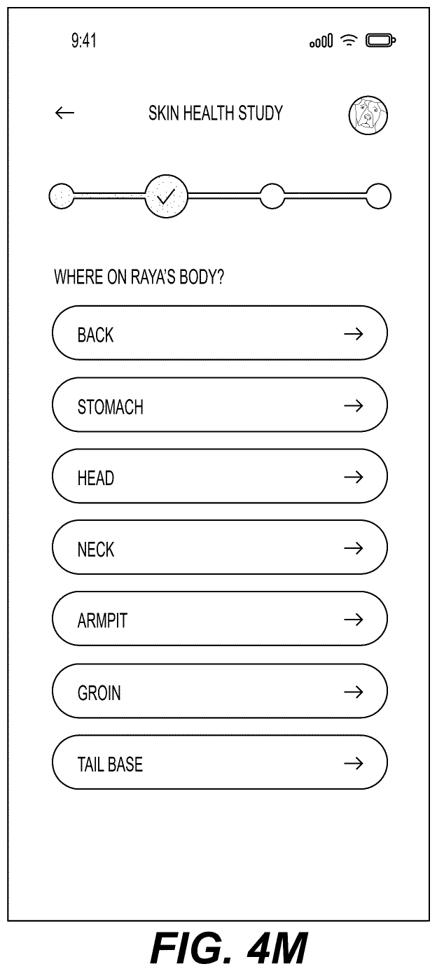

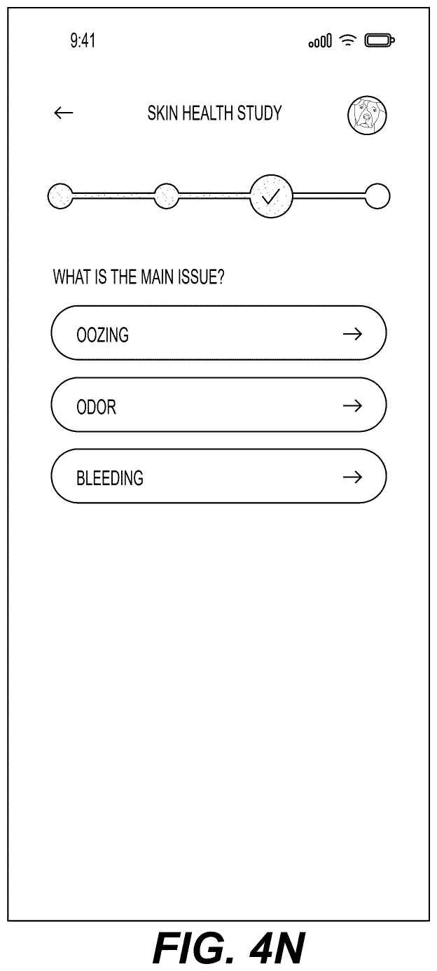

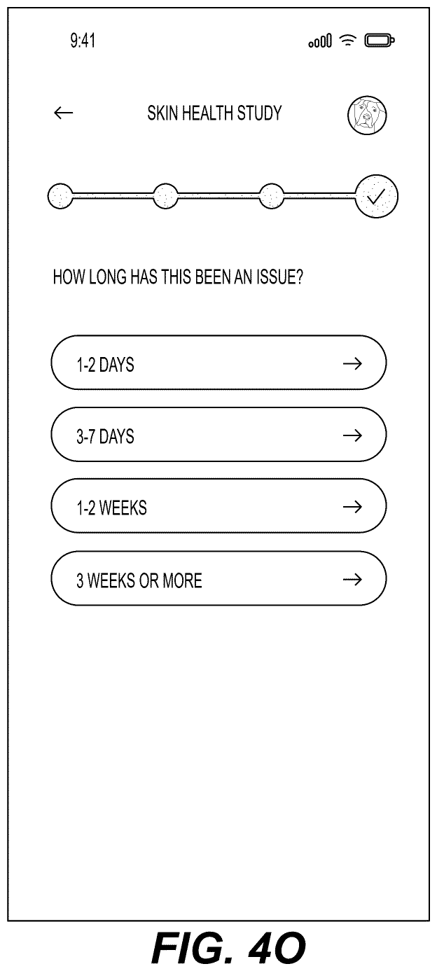

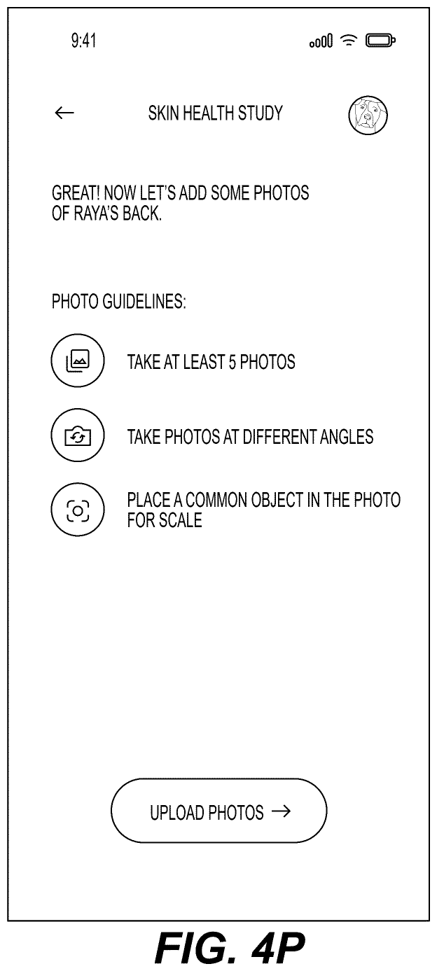

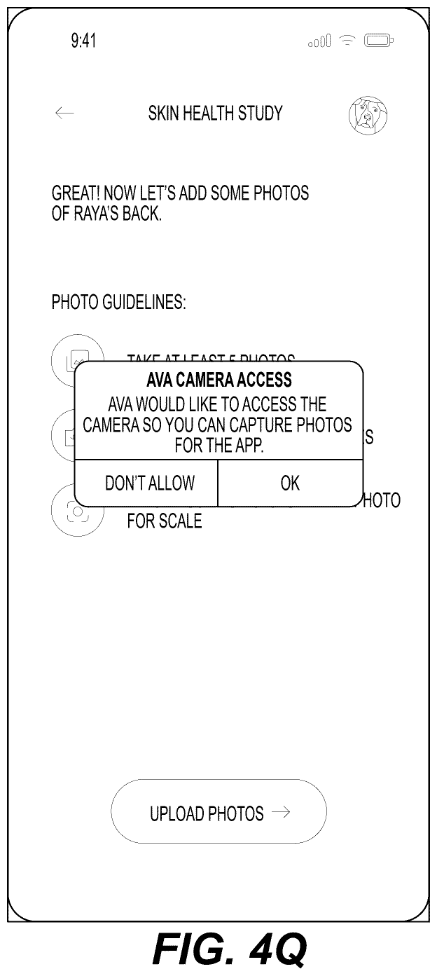

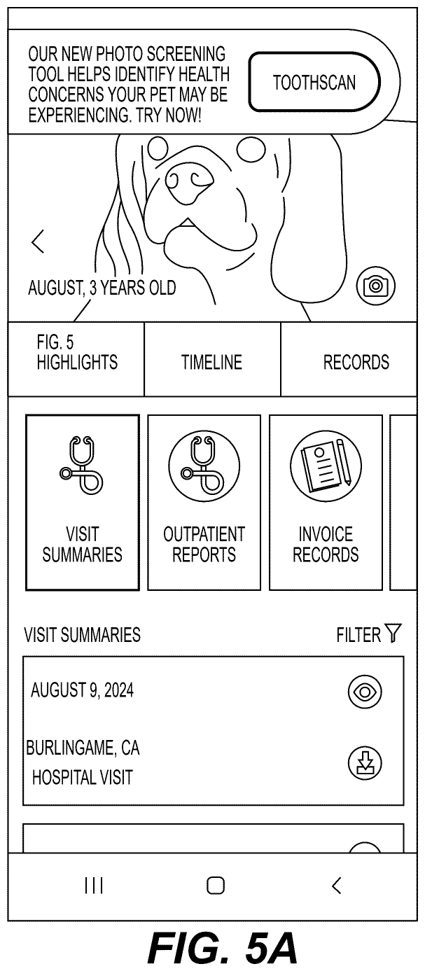

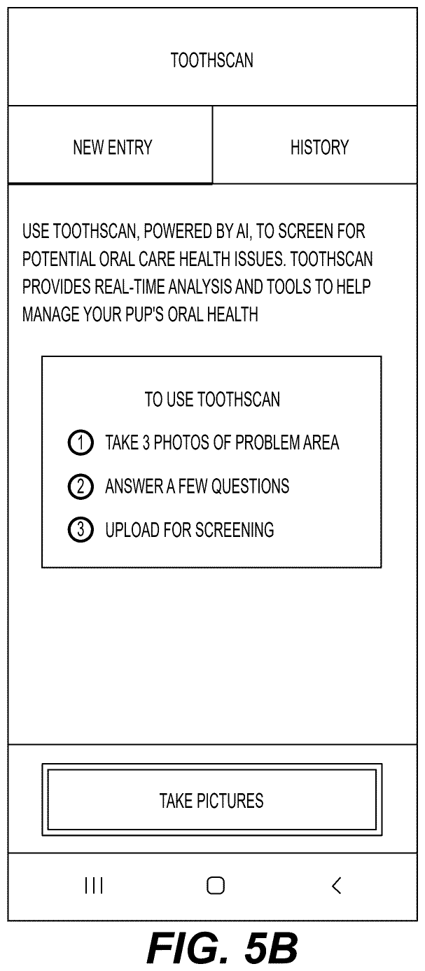

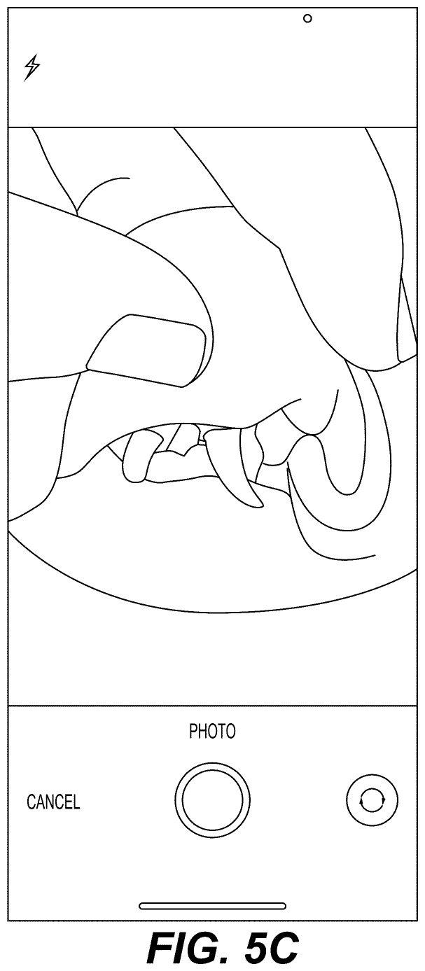

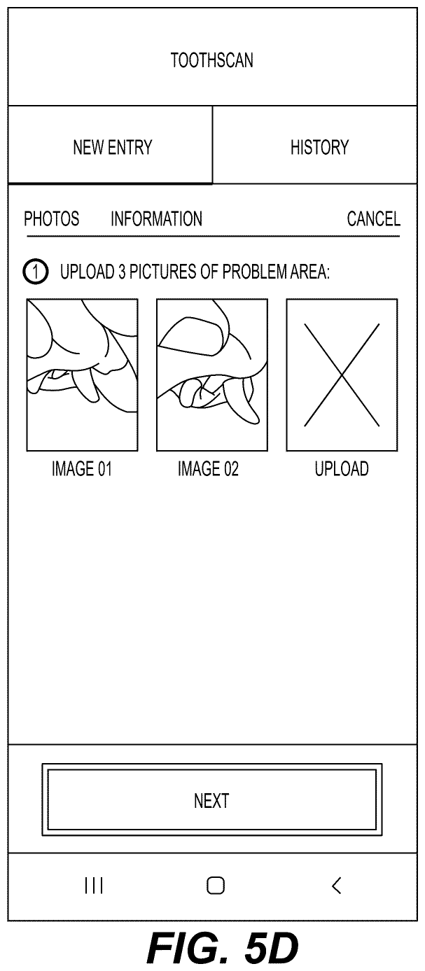

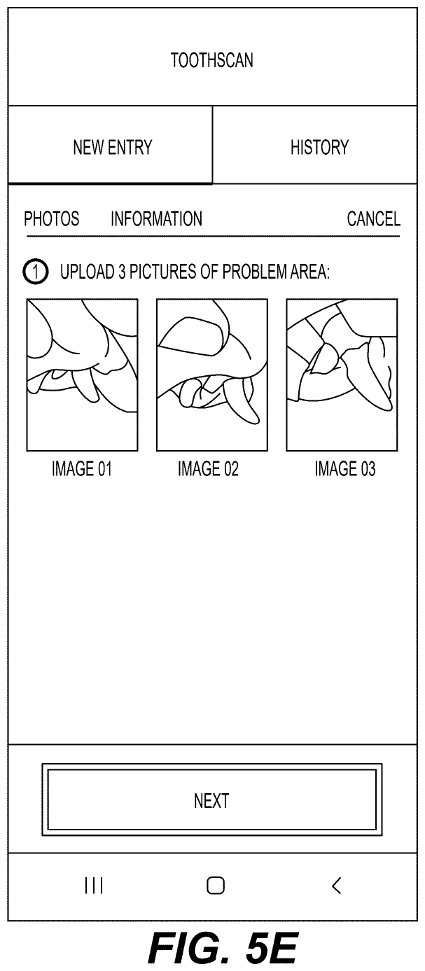

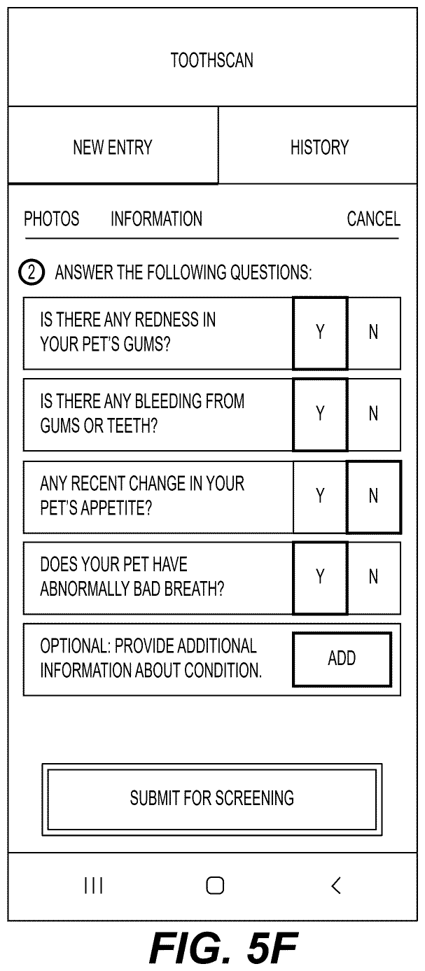

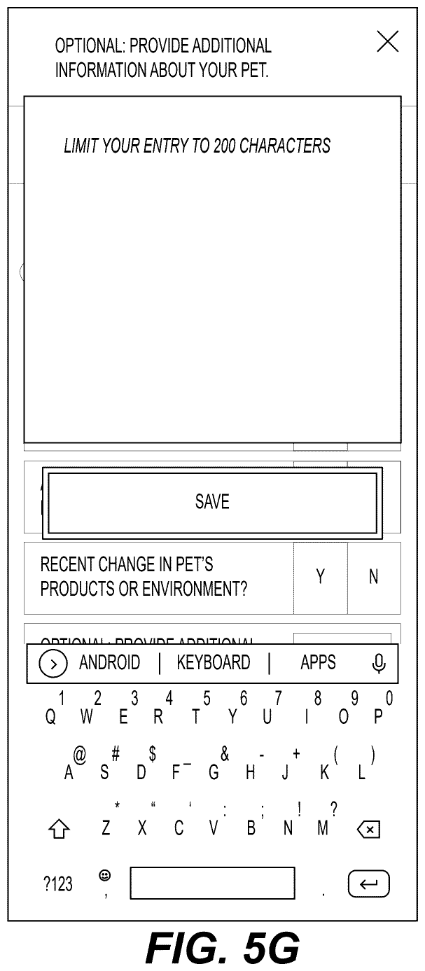

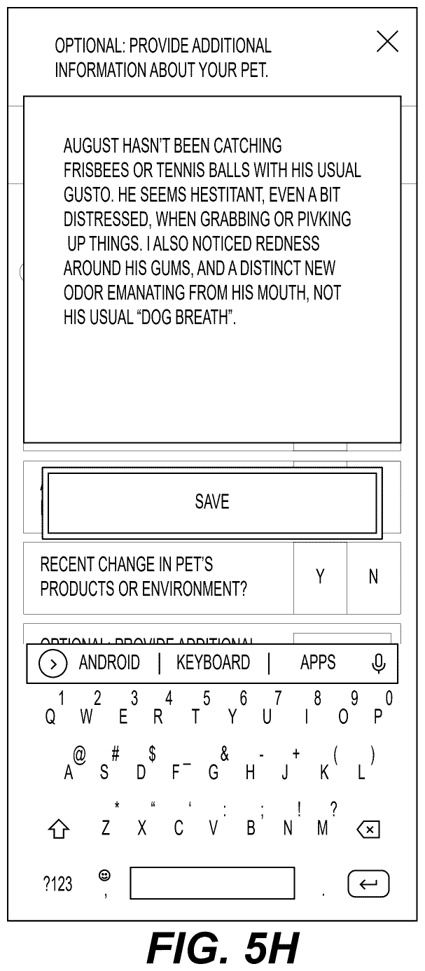

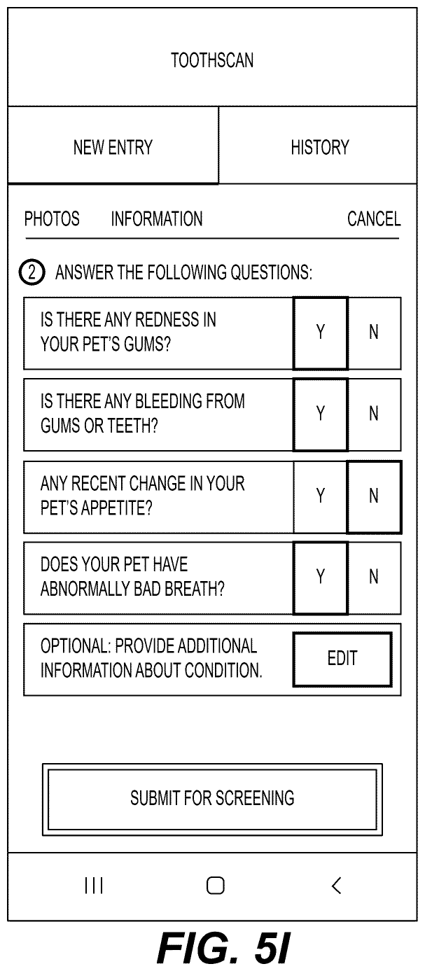

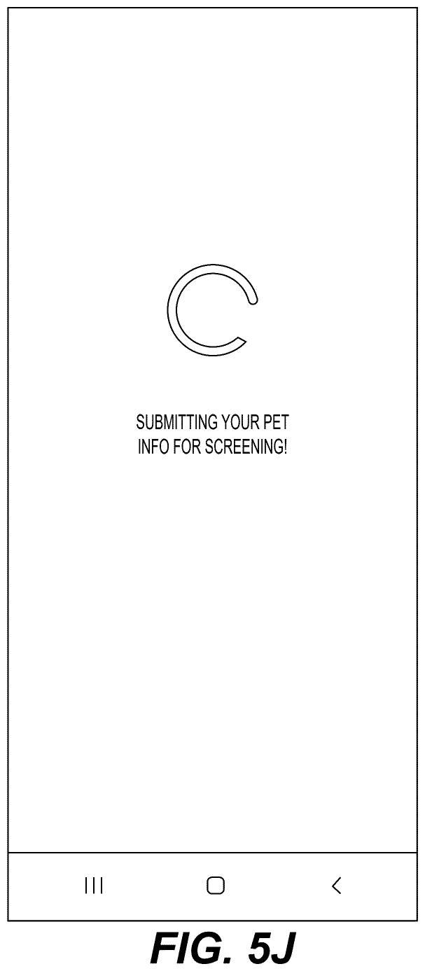

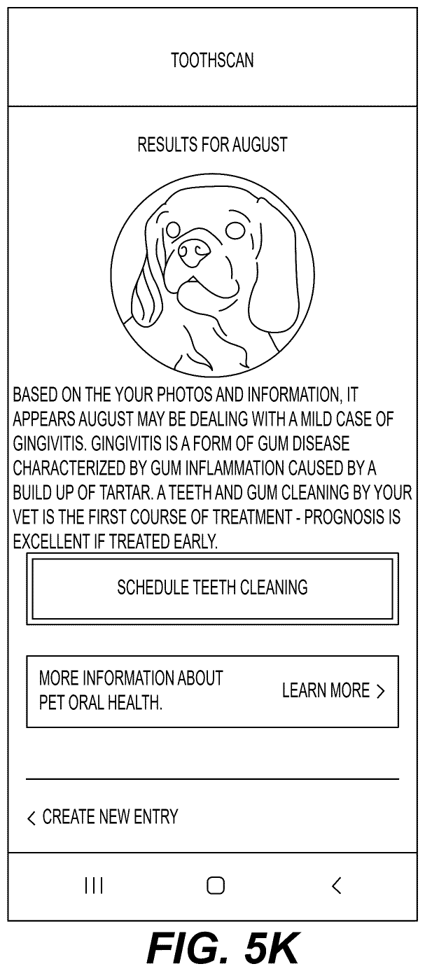

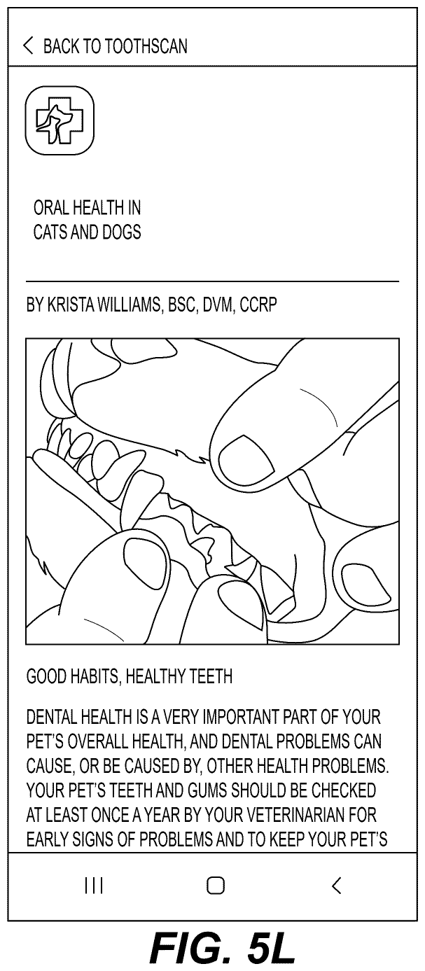

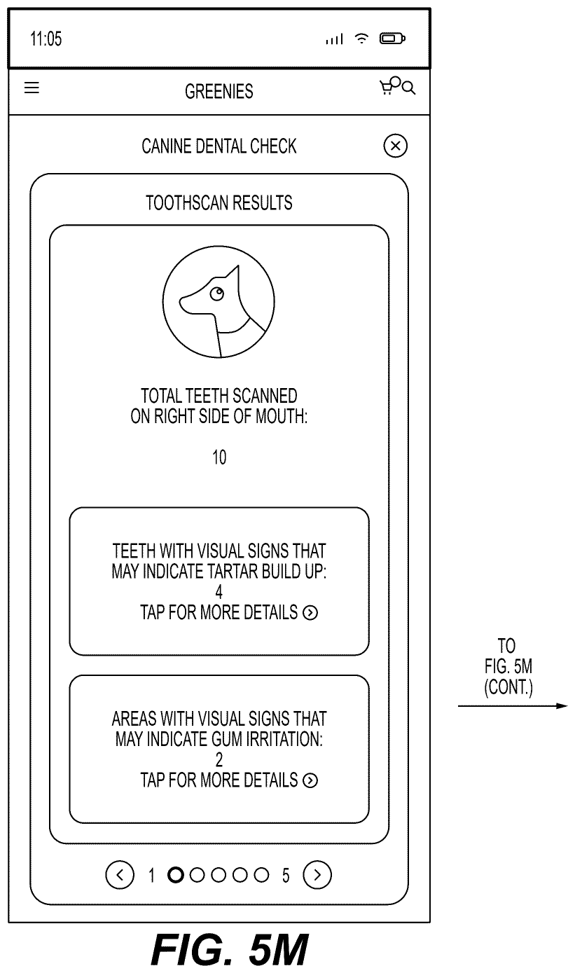

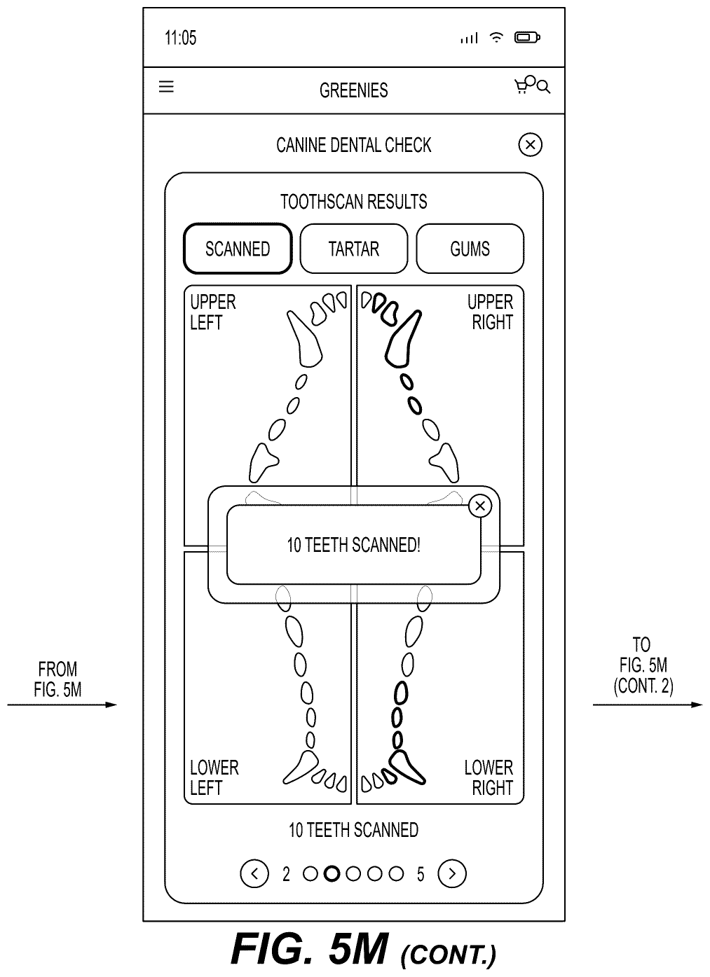

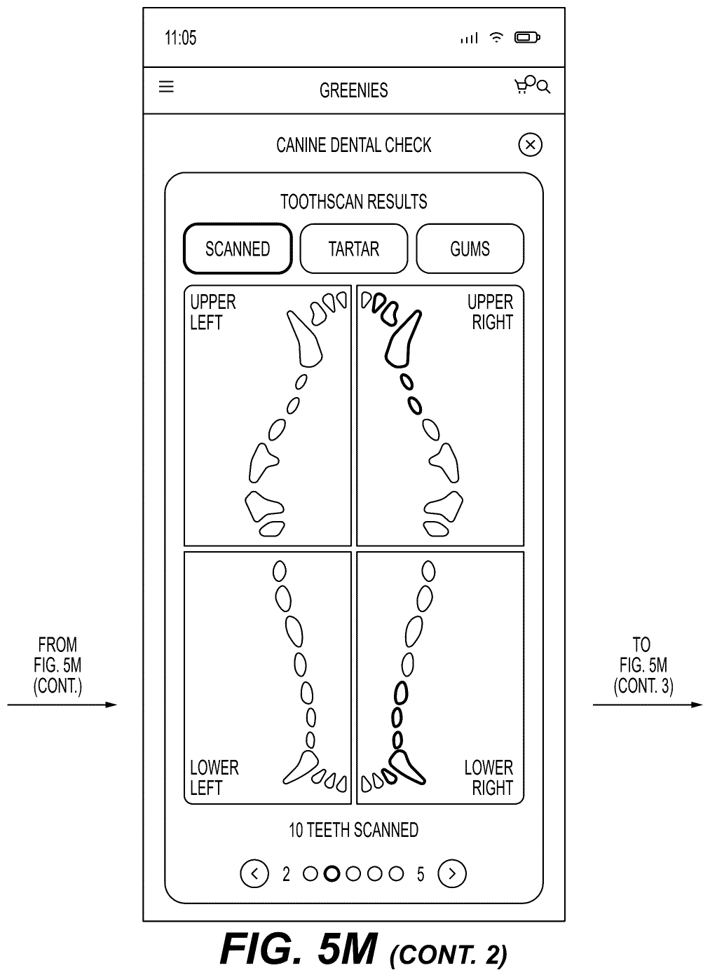

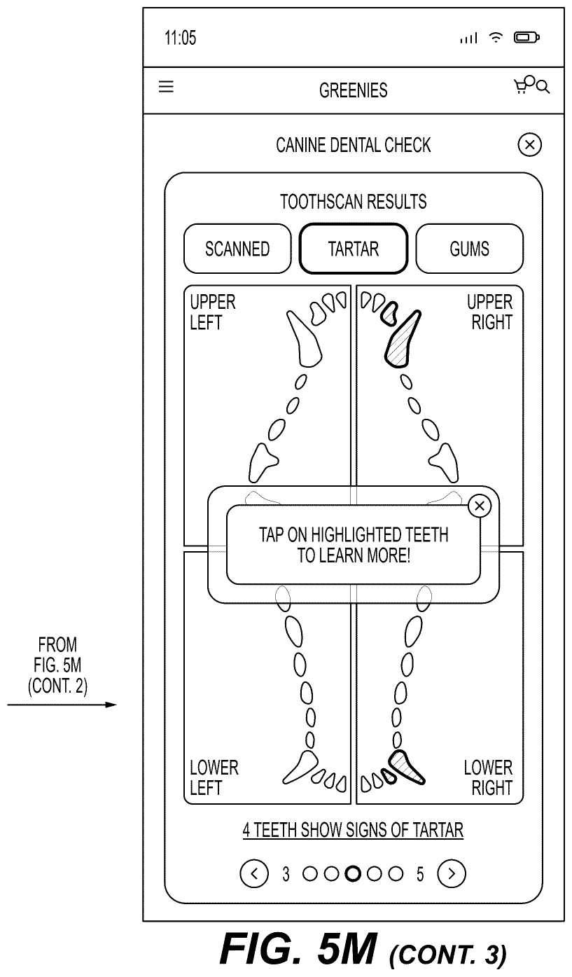

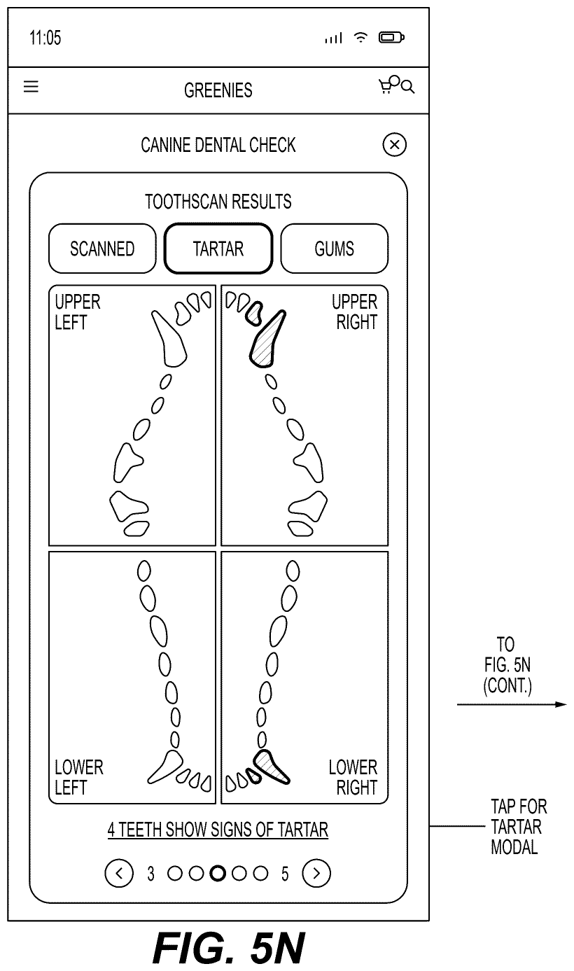

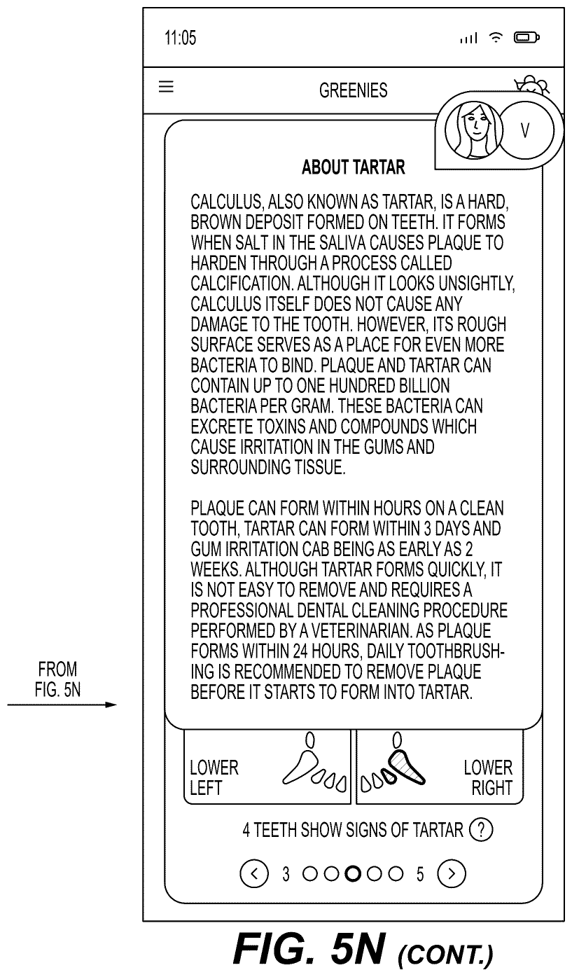

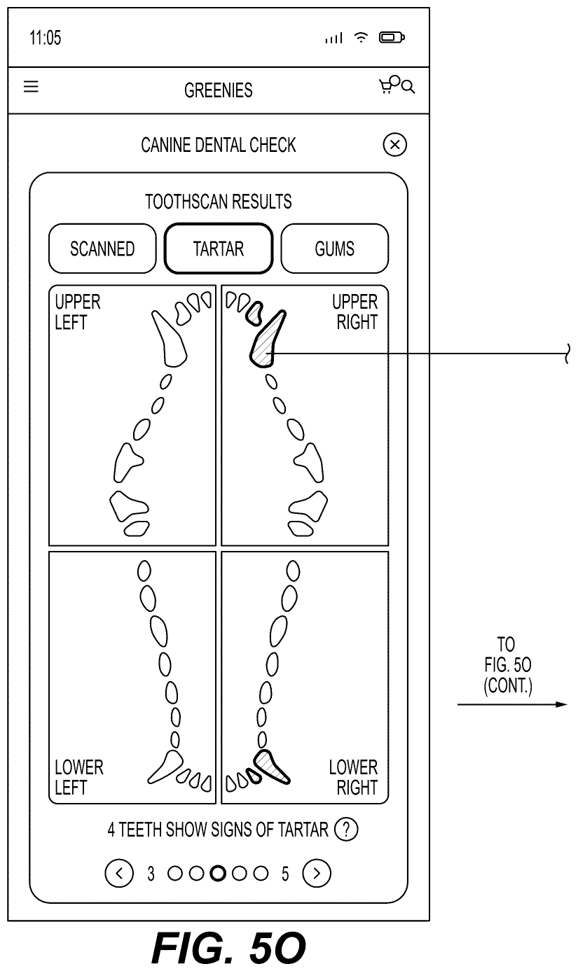

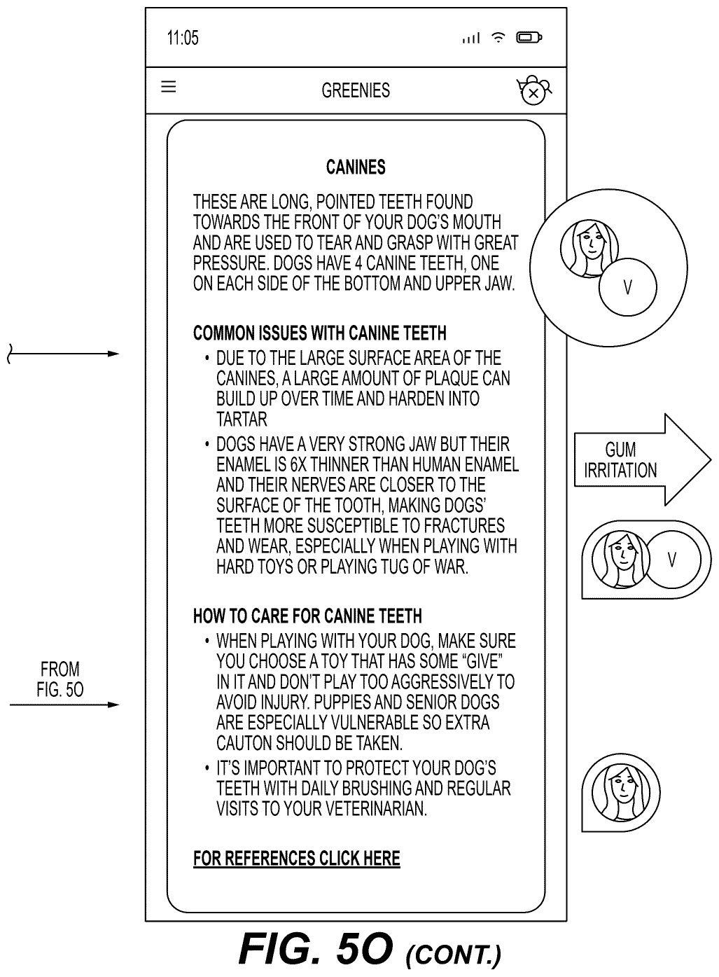

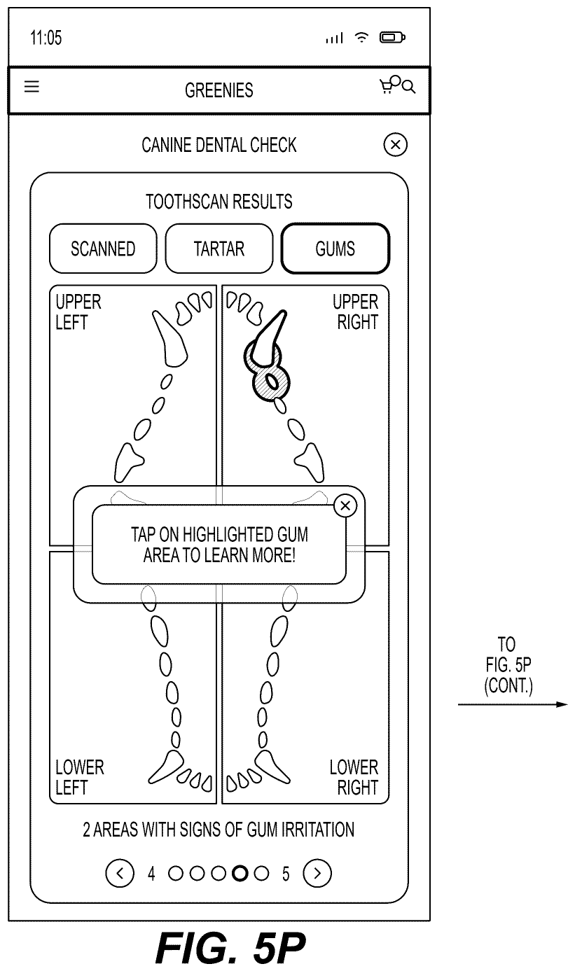

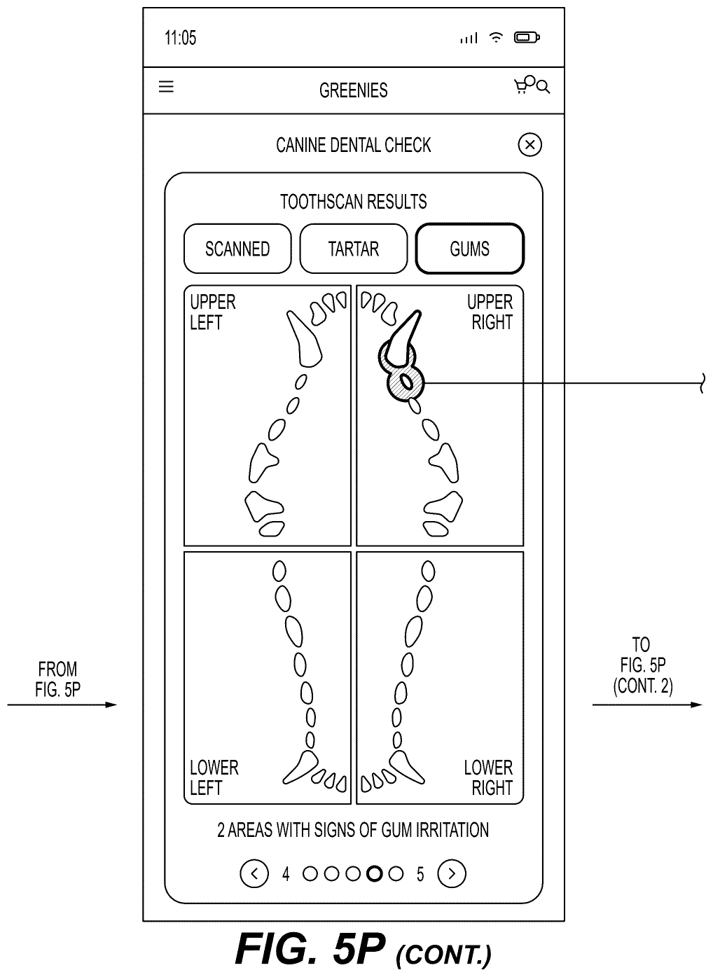

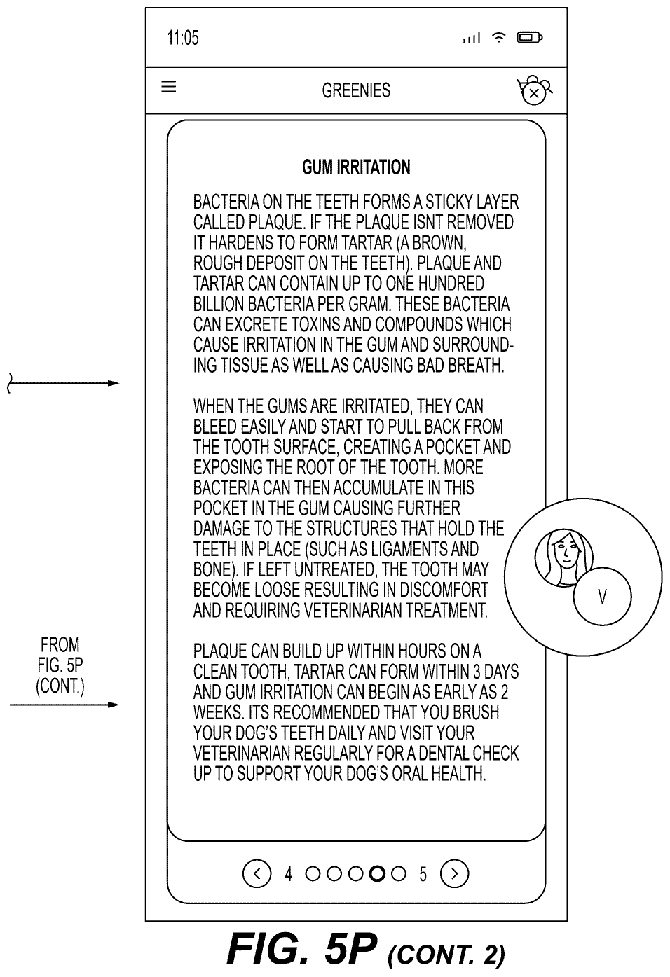

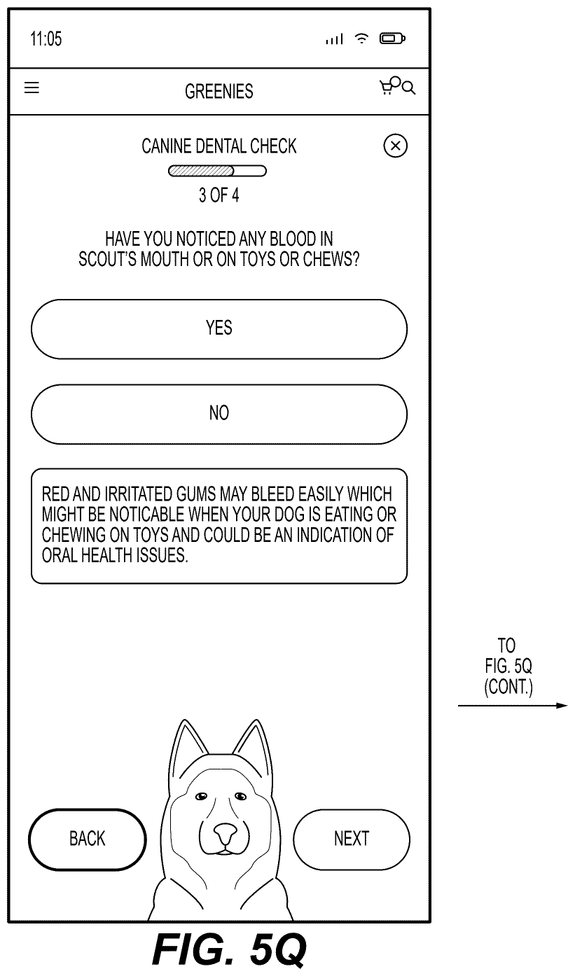

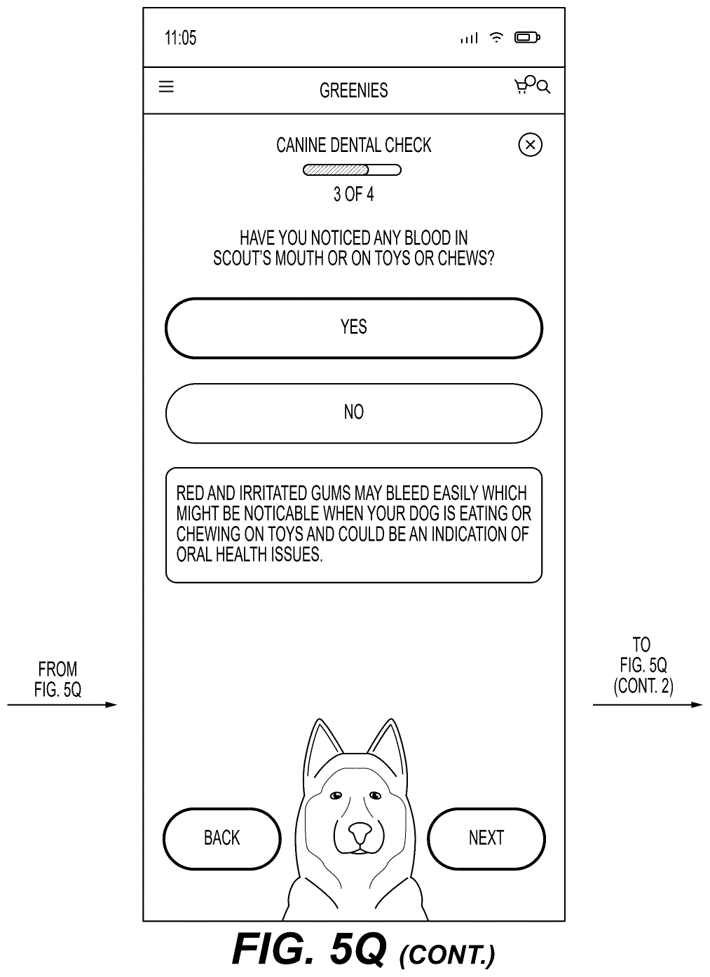

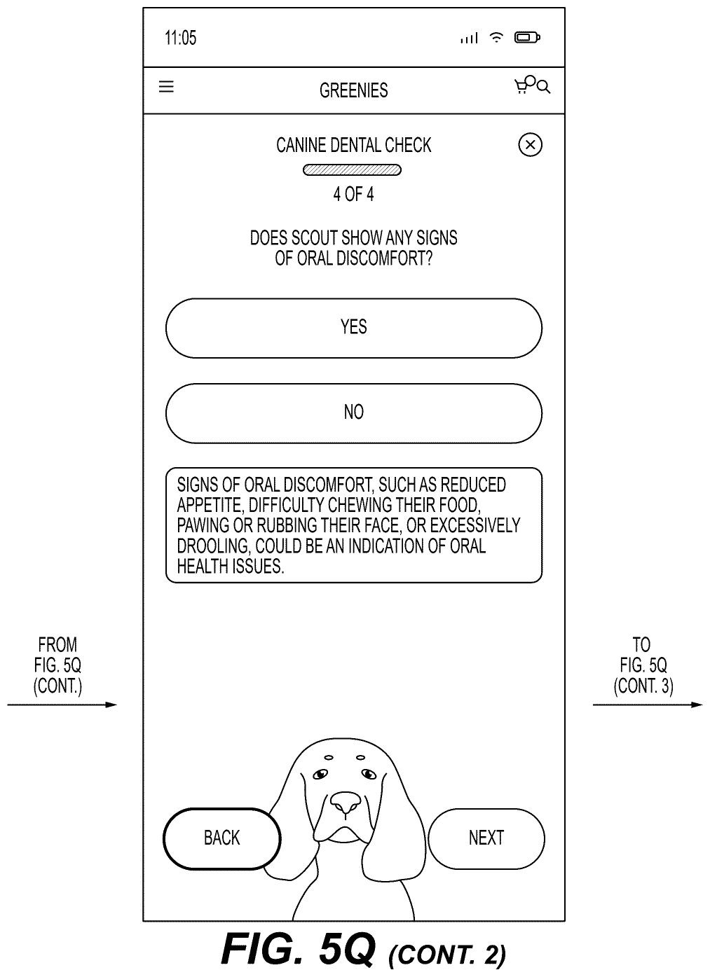

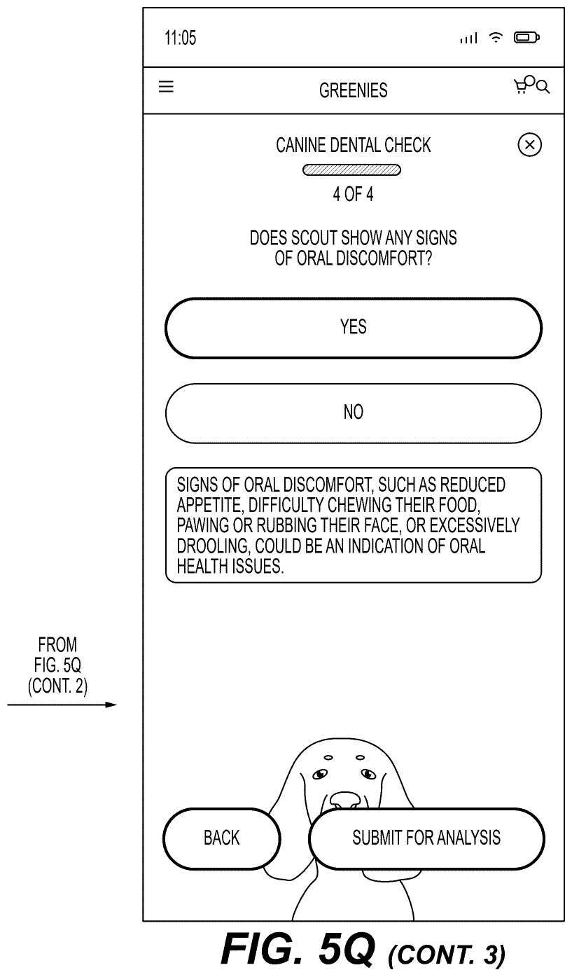

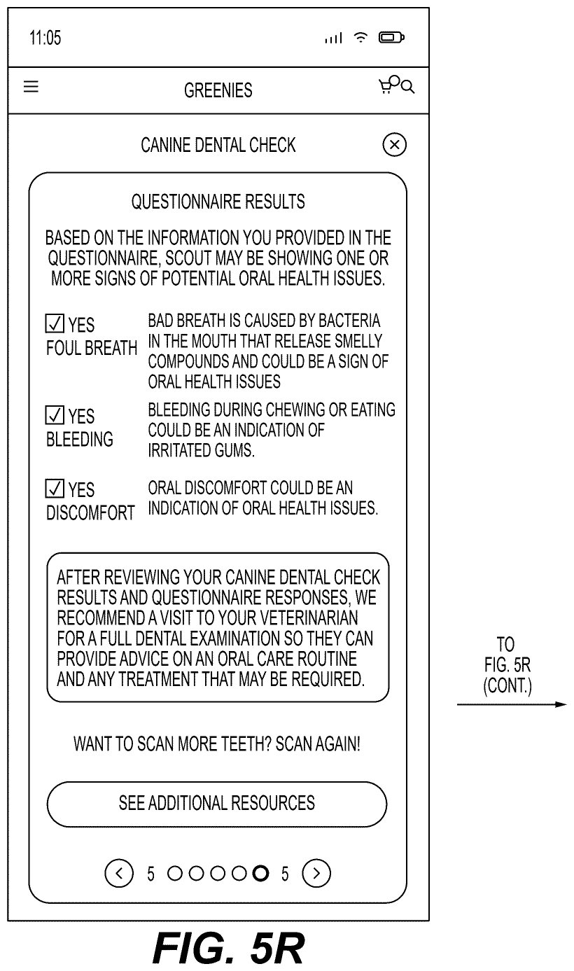

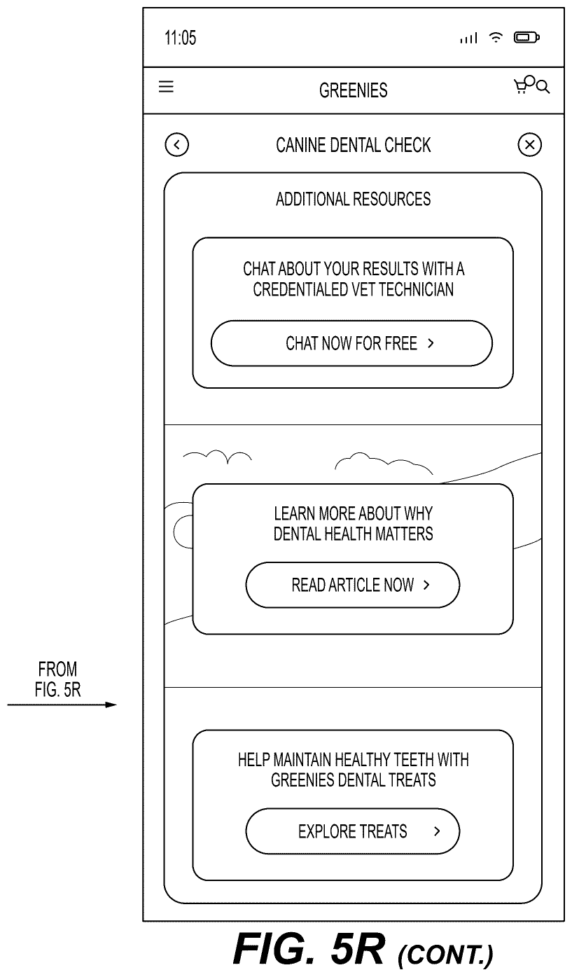

The diagnostic system 142 may manage a pet's health information and provide personalized diagnostics 144 and/or a personalized wellness plan 146 to the user. The diagnostic system 142 may be an internal component or an external component of platform 102, where the diagnostic system 142 may communicate with platform 102 via APIs 106. For example, the diagnostic system 142 may include a mobile application for utilizing an image classifier, as described in FIGS. 4A-Q and FIGS. 5A-L.

The diagnostic system 142 may manage a pet's heath information (e.g., vaccination records, medical records) by receiving the pet's health information from one or more external services 150 (e.g., veterinarians, clinics, pet hospital, pharmaceutical companies, and the like). The diagnostic system 142 may store the pet's health information in the pet profile(s) 118. In an embodiment, the diagnostic system 142 may communicate the pet's health information using APIs 106.

The diagnostic system may create personalized diagnostics 144 and/or a personalized wellness plan 146 based on the pet's health information. The personalized diagnostics 144 may include one or more diagnoses (e.g., ear infection, eye infection, and the like) of medical conditions for the pet. The personalized diagnostics 144 may be based on diagnoses made by the external services 150. In some embodiments, the personalized diagnostics 144 may be based on diagnoses made by one or more machine learning models. The personalized wellness plan 146 may include one or more recommendations regarding eating events, exercise events, health checks and wellness visits, and the like, which may be based on the pet's heath information. The personalized wellness plan 146 may be based on recommendations made by the external services 150. The personalized wellness plan 146 may be based on information included in the pet profile(s) 118. In some embodiments, the personalized wellness plan may be based on one or more recommendations made by one or more machine learning models. For example, as described further, the recommendations may correspond to recommendations to address a pet's oral condition or dermatological condition.

The health portal 148 may provide access to one or more parties who wish to retrieve the personalized diagnostics 144, personalized wellness plan 146, and/or the pet's health information from the pet profile(s) 118. The health portal 148 may be internal or external to the diagnostic system 142. Additionally, the health portal 148 may include a user interface. For example, a groomer may access the health portal 148 to retrieve the pet's vaccination records from diagnostic system 142.

The diagnostic system 142 may communicate with external services 150, such as veterinarians, clinics, pet hospital, pharmaceutical companies, and the like. For example, an external service 150 (e.g., veterinarian) may send updated vaccine or medical records to the diagnostic system 142, where the diagnostic system 142 may then store such updated vaccine or medical records in the pet profile(s) 118. Additionally, for example, the diagnostic system may update the personalized diagnostics 144 and/or the personalized wellness plan 146 based on the updated vaccine or medical records.

In some embodiments, the diagnostic system 142 may receive and store the pet's vaccination and treatment information. For example, if a veterinarian administers a medication, vaccination, and/or alternative therapy to a pet, the external service(s) 150 may send the medical details to the diagnostic system 142, where the diagnostic system 142 may receive and store the medication, vaccination, and/or alternative therapy details (e.g., a medication dosage amount, a medication description, a medication administrator, and/or a medication administration timestamp). Additionally, in some embodiments, the diagnostic system 142 may store the medication details in the platform 102 (e.g., database(s) 104, cloud storage 114, and/or pet profile(s) 118). The diagnostic system 142 may also communicate the pet's vaccination and treatment information to the external service(s) 150. For example, the diagnostic system 142 may receive and store medication details from several external service(s) 142. The diagnostic system 142 may receive a request from one of the external service(s) 142 for the medication details of a particular pet. Upon receipt of the request, the diagnostic system 142 may communicate the medication details to the external service(s) 142.

In some embodiments, the diagnostic system 142 may include information to authenticate the pet. For example, social media websites frequently require that a user is authenticated in order to label the user as “verified” (e.g., a blue checkmark). The diagnostic system 142 may contain information corresponding to a physical examination of the pet. Such information may include authentication information of the pet. For example, the authentication information may include a confirmation of the pet's breed, gender, image, etc. Such authentication information may be used by a social media website to authenticate the pet as a “verified” user.

### Homing System

The homing system 152 may match a future pet owner with a pet and provide additional support for the future pet owner. The homing system 152 may be an internal component or an external component of platform 102, where the homing system 152 may communicate with platform 102 via APIs 106.

The homing system 152 may match a future pet owner with a particular pet using a personalized matching module 154 and/or a search engine 156. The personalized matching module 154 may use user information (e.g., user location, user age, and the like) from the future pet owner (e.g., from the pet owner profile 120) to automatically search for one or more pets that are best suited for the future pet owner. In some embodiments, the personalized matching module 154 may use one or more machine learning models to determine the best pet matches for the future pet owner. The search engine 156 may allow the future pet owner to search for one or more pets. The search engine 156 may include different search filters (e.g., filtering by breed, age, size, weight, and the like), which may allow the user to filter the results of the one or more pets.

Both the personalized matching module 154 and/or the search engine 156 may retrieve results from the external services 162. The external services 162 may include one or more of: a pet adoption agency, a shelter, a pet breeder, and the like. When the personalized matching module 154 and/or the search engine 156 is performing a search for one or more pets, the personalized matching module 154 and/or the search engine 156 may send one or more requests to the external services 162 for available pets that fit one or more parameters contained in the one or more requests. Upon receiving the one or more requests, the external services 162 may search one or more databases for one or more matching pets. The external services 162 may send a response to the personalized matching module 154 and/or the search engine 156. The response may include the one or more matching pets. Alternatively, for example, if no matching pets were found, the response may include an indicator that no matching pets were found. In some embodiments, the homing system 152 may store the one or more matching pets in a database, such as an internal database or an external database (e.g., database 104).

The homing system 152 may display the one or more matching pets to the future pet owner, along with an option for the future pet owner to adopt and/or purchase the one or more matching pets. The homing system 152 may also facilitate the adoption and/or purchase of the one or more matching pets. In some embodiments, the homing system 152 may communicate with the external services 162 to facilitate the adoption and/or purchase of the one or more matching pets.

Once the future pet owner purchases and/or adopts the pet, the homing system 152 may store and/or manage the pet's adoption/registration record 160. In some embodiments, the homing system 152 may receive all (or part of) the pet's adoption/registration record 160 from the external services. In some embodiments, the homing system 152 may store the pet's adoption/registration record 160 in the pet profile(s) 118. Additionally, or alternatively, the homing system 152 may store the pet's adoption/registration record in the pet owner profile 120. In some embodiments, the homing system 152 may store the pet's adoption/registration record 160 in an internal or external database.

The homing system 152 may provide additional support for the future pet owner by providing personalized recommendations 158 to the pet owner. The personalized recommendations 158 may be based characteristics of the pet that the future pet owner purchased and/or adopted. Example personalized recommendations 158 may include a recommended pet food, a recommended pet provider, recommended pet supplies, and the like. In some embodiments, the personalized recommendations 158 may be based on communications with one or more of the external services. For example, the homing system 152 may communicate with the content management system 164 to receive personalized content 168, and then make personalized recommendations 158 based on the personalized content 168.

### Content Management System

The content management system 164 may provide personalized content 168 to a user. The content management system 164 may be an internal component or an external component of platform 102, where the content management system 164 may communicate with platform 102 via APIs 106.

The content management system 164 may retrieve personalized content 168 and display such personalized content 168 to the user. The personalized content 168 may include at least one of: an article, a blog post, an online forum, an advertisement, and the like. The personalized content 168 may also include recommendations that are specific towards the pet and/or user. The recommendations may include food recommendations, activity recommendations, product recommendations, resource recommendations (e.g., books, articles, and the like), third party services recommendations (e.g., groomer, trainer, boarding), and the like. The personalized content 168 may be personalized based on pet profile(s) 118 and/or pet owner profile 120. The content management system 164 may display the personalized content 168 via a user interface of a user device. In some embodiments, the content management system 164 may retrieve the personalized content 168 from the external services 166. The external services 166 may include an electronic magazine, one or more databases, one or more social media posts, and the like. In some embodiments, the content management system 164 may retrieve the personalized content 168 from other sources, such as database(s) 104, cloud storage 114, and personalized advertising system 108. In some embodiments, the content management system 164 may create personalized content 168 based on communications with the other external systems (e.g., wellness system 124, diagnostic system 142, homing system 152, genetics system 170, third party services system 182, etc.). For example, the content management system 164 may receive the personalized wellness plan 146 from diagnostic system 142. The personalized content 168 may then be based on (or include) information from the personalized wellness plan 146.

### Genetics System

The genetics system 170 may analyze and/or monitor a pet's genetic data. The genetics system 170 may be an internal component or an external component of platform 102, where the genetics system 170 may communicate with platform 102 via APIs 106.

The genetics system 170 may include genetic data analysis 172, genetic data monitoring 174, and/or personalized recommendations 176. Additionally, the genetic data analysis 172 and/or the genetic data monitoring 174 may communicate with external services 180 to assist with the analysis and/or the monitoring of the genetic data. The external services may include a laboratory, a clinic, a veterinarian, and the like.

The genetic data analysis 172 may receive genetic data belonging to a pet. In some embodiments, the genetic data analysis 172 may receive the genetic data from a genetic data retrieval system 178. The genetic data retrieval system 178 may retrieve and store genetic data belonging to one or more pets. Additionally, the genetic data analysis may receive genetic data from the genetic data retrieval system 178, where the received genetic data is used in the analysis of the genetic data belonging to the pet. The genetic data analysis 172 may analyze the genetic data to determine abnormalities, potential genetic traits, familial relationships, and the like. In some embodiments, the genetic data analysis 172 may communicate with external services 180 to assist with the analysis of the genetic data. For example, the genetic data analysis 172 may send genetic data information to a laboratory for the laboratory to perform the analysis of the genetic data.

The genetic data monitoring 174 may monitor the genetic data belonging to a pet to determine any changes in the genetic data. For example the genetic data monitoring 174 may receive new genetic data and compare the new genetic data to previously stored genetic data. The comparing may lead the genetic data monitoring 174 to determine that there is an abnormality or an improvement in the genetic data. In some embodiments, the genetic data monitoring 174 may communicate with the external services 180, in order for the external services 180 to analyze the genetic data and determine if there are any changes.

The genetics system 170 may provide personalized recommendations 176 to the user. For example, the genetics system 170 may provide personalized recommendations 176 to the user via a user interface of a user device. In some embodiments, the personalized recommendations may be based on the genetic data analysis 172 and/or the genetic data monitoring 174. The personalized recommendations 176 may include a pet food recommendation, an exercise recommendation, a pet item recommendation, health checks or wellness visits, and the like. In some embodiments, the personalized recommendations 176 may be based on communications with one or more of the external services. For example, the genetics system 170 may communicate with the diagnostic system 142. The genetics system 170 may send a request to the diagnostic system 142 for a personalized wellness plan 146. The request may include, for example, the genetic data analysis 172 and/or the genetic data monitoring 174. The diagnostic system 142 may communicate a personalized wellness plan 146 to the genetics system 170, where the personalized wellness plan 146 may be based on the genetic data analysis 172 and/or the genetic data monitoring 174. The genetics system 170 may make personalized recommendations 176 to the user based on the personalized wellness plan 146.

In some embodiments, the genetics system 170 may include information to authenticate the pet. For example, social media websites frequently require that a user is authenticated in order to label the user as “verified” (e.g., a blue checkmark). The genetics system 170 may contain information corresponding to a physical examination of the pet. Such information may include authentication information of the pet. For example, the authentication information may include a confirmation of the pet's breed, gender, image, etc. Such authentication information may be used by a social media website to authenticate the pet as a “verified” user.

### Third Party Services System

The third party services system 182 may allow a user to search for and reserve different external services 190, such as groomers, trainers, veterinarians, holistic care (e.g., nutritionist, naturopathic), and the like. The third party services system 182 may be an internal component or an external component of platform 102, where the third party services system 182 may communicate with platform 102 via APIs 106.

The third party services system 182 may include a search engine 184, a booking engine 186, and/or a management component 188.

The search engine 184 may allow the user, such as a pet owner, to search for external services 190 to reserve for the user's pet. The search engine 184 may include filtering functionality to facilitate a fine-tuned search. The filtering functionality may include universal filtering and/or service specific filtering. For example, the universal filtering may include filtering the external services 190 by location, price range, and/or ratings. Additionally, for example, the service specific filtering may include filtering the external services 190 by breed specialty, health issues, and/or behavioral needs.

The booking engine 186 may allow the user to reserve the external services 190. For example, after using the search engine 184 to search for external services 190, the user may use the booking engine 186 to reserve a particular service of the external services 190. The booking engine 186 may present open dates and time slots, which may correspond to the selected external service 190. The user may then user the booking engine 186 to select a date and/or time from the displayed open dates and time slots. Upon the finalization of the booking, the user may receive an instant confirmation of the booking, such as via text or email. The user may also have the ability to instantly pay for the booked service. Alternatively, the user may be able to pay upon the finalization of the service. The user may be able to upload photos and include notes to the external service 190. For example, the user may upload dog photos to a groomer, or make a note that the user's dog has a limp.

The management component 188 may provide functionality to manage different external services 190. For example, the management component 188 may provide the functionality for external services 190 to register and/or be removed from the third party services system 182. The management component 188 may communicate with one or more databases (e.g., database(s) 104) and/or cloud storage (e.g., cloud storage 114) to store information (e.g., a name, a business identifier, a specialty, and the like) corresponding to the external services 190.

FIG. 2 depicts an exemplary environment 200 of a pet owner profile 202 and corresponding pet profiles that may be utilized with the techniques presented herein. Notably, exemplary platform environment 200 may complement exemplary platform environment 100, with pet owner profile 202 corresponding to pet owner profile 120 of FIG. 1. Additionally, pet profile 204, pet profile 206, and/or pet profile 208 may correspond to pet profiles(s) 118 of FIG. 1.

Pet owner profile 202 may include at least one of: a pet owner name 210, a pet owner identifier 212, a pet owner address 214, a pet owner digital wallet 216, pet owner demographic information 218, a pet owner email address 220, at least one pet profile (e.g., pet profile 204, pet profile 206, pet profile 208) and/or at least one identifier associated with the at least one pet profile, and/or a pet owner history 222. The pet owner name 210 may include a name of the pet owner. The pet owner identifier 212 may include a unique identifier that may be used to locate the pet owner profile 202. In some embodiments, the pet owner identifier 212 may allow for tracking of some or all of the user's activities. The pet owner address 214 may include a physical address of the pet owner. The pet owner digital wallet 216 may include payment information, such as credit card information, cryptocurrency information, and the like. The pet owner demographic information 218 may include a particular demographic of the pet owner. The pet owner email address 220 may include an email address of the pet owner. The pet owner profile may include at least one pet profile (e.g., pet profile 204, pet profile 206, pet profile 208). In some embodiments, in lieu of including an entirety of the at least one pet profile, the pet owner profile 202 may include at least one identifier associated with the at least one pet profile (e.g., unique pet identifier 228). Each of the pet profiles may correspond to a pet that belongs to the pet owner. The number of pet profiles may be dynamic, where the pet profiles may adjust according to the number of pets that belong to the user.

The pet owner history 222 may include a payment history 224 and/or a booking history 226. The payment history 224 may include financial transactions of the pet owner. In some embodiments, the payment history 224 may correspond to activity of the pet owner digital wallet 216. In some embodiments, the payment history 224 may be tracked and analyzed to provide for targeted advertising (e.g., of personalized advertising system 108) and/or recommendations to the pet owner. The booking history 226 may include previous bookings of third party services that were made by the user. In some embodiments, the booking history 226 may be tracked and analyzed to provide for targeted advertising (e.g., of personalized advertising system 108) and/or recommendations to the pet owner.

Pet profile 204, pet profile 206, and/or pet profile 208 may each correspond to a different pet that belongs to the pet owner of the pet owner profile 202. The pet owner may have more or less than three pets. The number of pet profiles may be dynamic, where the number of pet profiles corresponds to the number of pets that belong to the pet owner. In some embodiments, the pet owner may want only a subset of the pet owner's pets to have pet profiles.

Pet profiles 204, 206, and/or 208 may each include at least one of: a unique pet identifier 228, breed/DNA information 230, veterinarian history 232, microchip information 234, a pet image 236, vaccination records 238, a purchase history 240, an adoption/registration record 242, activity data 244, a wellness score 246, an insurance policy 248, a wellness plan 250, a booking history 252, a pet name 254, medication history 256, dietary needs 258, and/or a pet savings account 260.

The unique pet identifier 228 may include a unique identifier that may be used to locate the corresponding pet profile (e.g., pet profiles 204, 206, and/or 208). In some embodiments, the unique pet identifier 228 may allow for tracking of some or all of activities corresponding to the pet.

The pet image 236 may include to a photograph, drawing, virtual presence, and/or avatar of the pet. The pet name 254 may include the name of the pet and/or any nicknames. The insurance policy 248 may include a pet insurance policy for the pet. The purchase history 240 may include purchases made for the pet. The pet savings account 260 may include a financial savings account for the pet. In some embodiments, the pet image 236, the pet name 254, the purchase history 240, pet savings account 260, and/or the insurance policy 248 may have been received from one or more of the external systems.

The breed/DNA information 230 may correspond to the breed and/or DNA information of the pet. In some embodiments, the breed/DNA information 230 may have been received from one or more of the external systems. For example, the breed/DNA information 230 may have been received from genetics system 170.

The veterinarian history 232 may include the details of the pet's visit(s) to a veterinarian. The veterinarian history 232 may also include notes from the vet and/or possible diagnoses and treatments. The vaccination records 238 may include one or more vaccination records of vaccinations administered to the pet. The medication history 256 may include details of the medications that the pet currently takes and/or has taken in the past. The dietary needs 258 may include information regarding food that the pet should eat and/or food that the pet should avoid. The wellness plan 250 may correspond to a wellness plan for the pet. In some embodiments, the wellness plan 250 may have been determined based on personalized wellness plan 146. In some embodiments, the veterinarian history 232, vaccination records 238, dietary needs 258, wellness plan 250, and/or the medication history 256 may have been received from one or more of the external systems. For example, the veterinarian history 232, vaccination records 238, dietary needs 258, wellness plan 250, and/or the medication history 256 may have been received from diagnostic system 142.

The microchip information 234 may include a microchip number of the pet. For example, the microchip may have been inserted into the pet to track the pet. The adoption/registration record 242 may include documentation of the adoption or purchase of the pet. In some embodiments, the microchip information 234 and/or adoption/registration record 242 may have been received from one or more of the external systems. For example, the microchip information 234 and/or adoption/registration record 242 may have been received from homing system 152.

The activity data 244 may include data corresponding to mobility data, physical activities, sleep activities, and/or food activities of the pet. For example, the mobility data and/or activity data may be collected by a smart collar 130, a smart bed 132, a smart feeder 134, a smart litter box 136, a smart camera 138, and/or the other sensors for collecting a digital image of a pet's life 140. The wellness score 246 may include data corresponding to a wellness score produced by wellness index scoring system 128. In some embodiments, the activity data 244 and/or the wellness score 246 may have been received from one or more of the external systems. For example, the activity data 244 and/or the wellness score 246 may have been received from wellness system 124.

The booking history 252 may include data corresponding to one or more bookings of a third party service (e.g., groomer, trainer, and the like). In some embodiments, the booking history 252 may have been received from one or more of the external systems. For example, the booking history 252 may have been received from the third party services system 182.

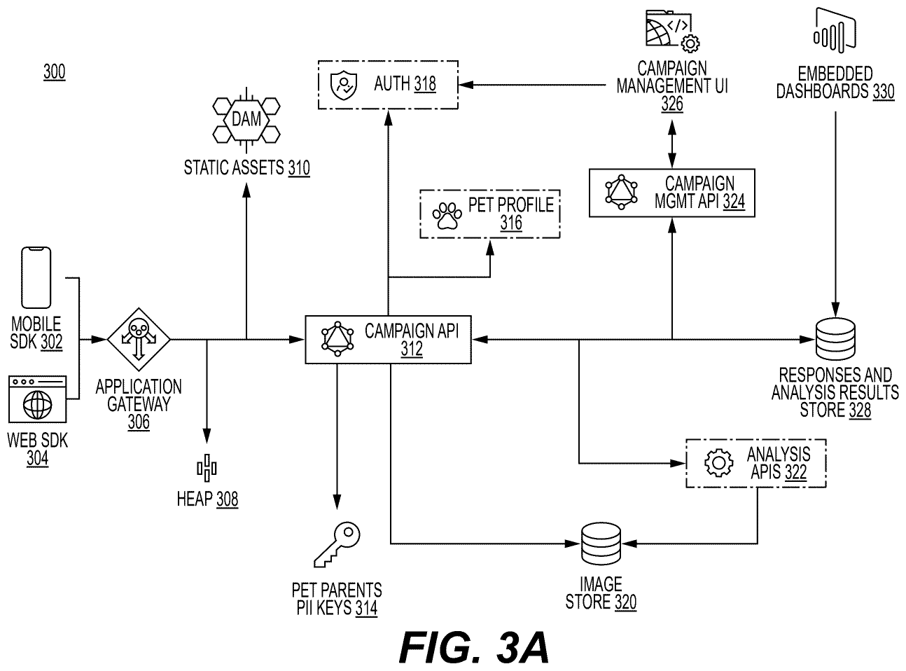

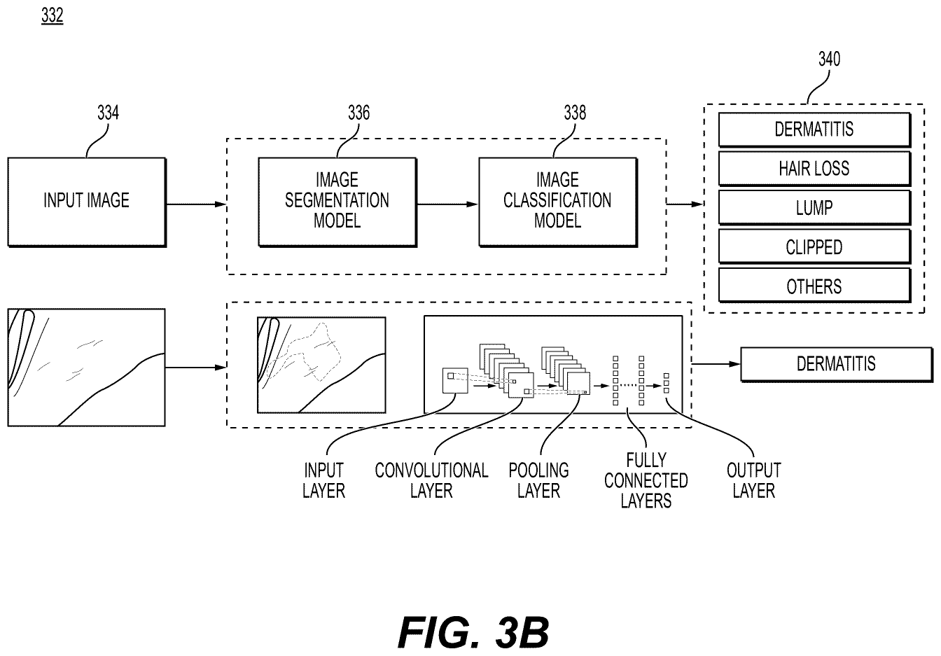

FIG. 3A depicts a flow chart of an exemplary campaign system 300, according to one or more embodiments. The campaign system 300 may be directed towards encouraging users (e.g., pet owners) to upload their pet photos (e.g., pet image data) and health condition information (e.g., pet data) to the image classifier. The image classifier may analyze the uploaded data to determine a pet condition and/or a corresponding recommendation. Additionally, or alternatively, the pet photos and/or the health condition information may be stored for training a machine-learning model. The campaign system may also include the functionality to adapt to other systems, such as visually adapting to other systems, where the functionality may remain consistent.

As shown in FIG. 3A, a mobile SDK 302 and/or a web SDK 304 may access an application gateway 306. The application gateway 306 may correspond to the image classifier. The campaign system 300 may include a heap 308 for efficient memory management and improved performance, especially in client-side operations and static assets 310 like HTML, CSS, JavaScript, images, and fonts that are served directly to the client without modification to provide fast loading times. The campaign API 312 may provide access to pet parent's PII (e.g., PII keys 314), a pet profile 316, authentication functions 318, an image store 320 (e.g., a database), and/or a responses and analysis results store 328 (e.g., a database). For example, the campaign API 312 may access/communicate with one or more external systems (e.g., external services 122). The image store 320 may store the pet's photos (e.g., pet image data). The responses and analysis results store 328 may store the uploaded pet data, the pet condition, and/or the recommendation. The responses and analysis results store 328 may interact with the image store 320 via one or more analysis APIs 322. The responses and analysis results store 328 may interact with one or more campaign management APIs 324, which may interact with a campaign management user interface 326. The campaign management user interface 324 may interact with an authentication module 318 of the campaign system 300. Additionally, or alternatively, one or more embedded dashboards 330 may interact with the responses and analysis results store 328.

FIG. 3B depicts a flow chart of an exemplary dermatological detection system 332, according to one or more embodiments. The dermatological detection system 332 may be directed towards analyzing pet images to determine a dermatological condition of the pet.

A user (e.g., pet owner) may input an image into the dermatological condition into the system (Step 334). The user may use a mobile device to capture a photo of the skin, teeth, tongue, and/or mouth of the pet. Alternatively, the user may select a stored photo of the device for upload to the system. For example, the photo may capture the pet's paw, fur/coat, tail, back, and the like.

An image segmentation model may receive the image, and then analyze the image to determine an abnormal region of the pet, e.g., the pet's skin (Step 336). The image segmentation model may have been previously trained to recognize and identify regions of the pet's skin that have an abnormal appearance. For example, identifying an abnormal region of the pet's skin may include highlighting and/or labeling the abnormal region. The image segmentation model may modify the image to identify the abnormal skin region. The modifying may include changing the contrast or brightness of the image and/or cropping the image to highlight the abnormal skin region.

The image segmentation model may input the image (e.g., modified to identify the abnormal skin region) into an image classification model, where the image classification model may be configured to analyze and classify the identified abnormal skin region (Step 338). The image classification model may include an input layer, a convolutional layer, a pooling layer, a fully connected layer(s), and/or an output layer. The input layer may receive the image from the image segmentation model, and then pass the image to the convolutional layer. The convolutional layer may be configured to identify feature maps (e.g., that correspond to the abnormal skin regions) in the image based on patterns and features in the images. The pooling layer may be configured to reduce the spatial dimensions of the feature maps. The connected layer(s) may be configured to classify the feature maps that have the reduced spatial dimensions. For example, the connected layer(s) may classify an abnormal skin region captured in the image. The output layer may be configured to output the classification of the abnormal skin region.

The classifications of the abnormal skin region may be output to a user interface of a user device (Step 340). Exemplary classifications of the abnormal skin region may include dermatitis, hair loss, one or more lumps, one or more clipped areas, and the like. Additionally, an abnormal skin region may include one or more classifications.

FIGS. 4A-Q depict an exemplary mobile application for utilizing an image classifier to collect image data to train one or more machine-learning models, according to one or more embodiments.

As shown in FIG. 4A, the mobile application may include an initial welcome page that may be output on a user interface of a user device. The welcome page may include a widget (e.g., “Get Started”), which when pressed, may initiate the pet condition analysis process for a pet. As shown in FIGS. 4B, 4C, and 4D, in response to initiating the pet condition analysis process, the mobile application may prompt the user to input pet data (e.g., “Pet Name,” “Breed,” “Age,” “Weight”). Additionally, as shown in FIG. 4E, in response to the prompts, the user may input pet data (e.g., “American French Bull Terrier,” “6 Years 2 Months,” “62 lbs”) via the user interface. As shown in FIGS. 4F, 4G, 4H, and 4I, the mobile application may prompt (e.g., “Add a Photo of Raya”) the user to input a photo of the user's pet, as well as access a camera/photo library of the user's device. As shown in FIGS. 4J and 4K, the mobile application may present the user with the option to upload image data (e.g., photos) to contribute to, for example, research studies for oral health or skin health. As shown in FIG. 4K, the user may select an option (e.g., “Join Study”) to join one or more of the studies. As shown in FIGS. 4L, 4M, 4N, and 40, the mobile application may present questions, which are specific to the particular study, regarding the pet's condition and symptoms. For example, the mobile application may ask questions regarding the general location (e.g., “Body,” “Paw,” “Leg”) of the condition on the pet's body. Based on the user's selection, the mobile application may display more detailed locations (e.g., “Back,” “Stomach,” “Head,” “Neck,” “Armpit,” “Groin,” “Tail Base”). The mobile application may also display questions regarding the pet's symptoms. For example, the mobile application may display questions regarding the main issue (e.g., “Oozing,” “Odor,” “Bleeding”). Upon selection of a main issue, the mobile application may also display questions regarding how long the pet has experienced the main issue (e.g., “1-2 Days,” “3-7 Days,” “1-2 Weeks,” “3 Weeks or More”). As shown in FIGS. 4P and 4Q, the mobile application may display prompts for the user to upload pet image data (e.g., photos). Upon receiving the pet image data, the mobile application may store the pet image data and/or the collected pet data in one or more databases. Machine-learning models may then access the stored pet image data and/or the collected pet data for training and tuning purposes.

The training process may include pre-processing the pet image data (e.g., that includes pet teeth and/or gums). In addition to image data that corresponds to the pet, the pet image data may further include annotations (e.g., labels) that correspond to whether a pet's tooth is normal or abnormal (e.g., tartar, gingivitis). For example, the images may be annotated with masks that cover the visible tooth and a label (e.g., “Healthy,” “Dental Calculi,” “Gingivitis,” “Gingival Recession”). In some embodiments, one or more attributes may be added to each mask, where each attribute may represent a specific disease. Exemplary attributes may include calculus, plaque, bruised, fractured, persistent, furcation, and/or non-diagnostic (e.g., teeth that are not clearly visible). Each tooth may have multiple attributes, in order to represent scenarios where a tooth may have multiple issues. Moreover, masks without any attribute may be implicitly considered as healthy by the machine-learning model.

Pre-processing the pet image data may be useful for increasing the efficiency of the system. For example, using high resolution images for machine-learning model training may slow down the training process, as using high resolution images may significantly reduce the batch size. For example, in some instances, the predictions from the machine-learning model may depend on storage that may exceed the available graphics processing unit (GPU) memory, resulting in killing the training process. To address such a scenario, the pet image data may be resized by maintaining the aspect ratio, such that the maximum smallest side of any image does not exceed a particular size (e.g., 1000 pixels). Moreover, the pet image data may also include As a result, resizing the pet image data may include resizing the annotations. The resized dataset of the pet image data may be used for training and validating the machine-learning model(s).

The training process may include dividing the annotated dataset to create a training dataset and a validating dataset. This process may include ensuring that all of the images from the same dog are added to either the training dataset or the validating dataset. During the training process, the machine-learning model may receive the training dataset, and in response to learning associations and patterns that are based on the training dataset. During the validation process, the machine-learning model may analyze the validating dataset to generate a prediction. The prediction may include a bounding box, a mask, and/or a label for one or more teeth and a corresponding predicted diagnosis.

FIGS. 5A-L depict an exemplary mobile application for utilizing an image classifier to determine a pet condition and a corresponding recommendation, according to one or more embodiments.

As shown in FIG. 5A, a mobile application for an external system may include an electronic user interface displaying a widget (e.g., “Toothscan”) of the image classifier mobile application. In response to a user interacting with or selecting the widget, the user interface of FIG. 5B may be displayed on the user's mobile device, which displays an initial user interface of the image classifier mobile application. The initial user interface may include a widget for allowing the user to upload pet image data (e.g., “Take Pictures”). As shown in FIG. 5C, the user may capture the pet image data by using a camera of the user device. As shown in FIGS. 5D-E, the user may upload multiple photos of the pet's problem area, e.g., of the pet's teeth, tongue, mouth, skin, etc. After uploading the image data, the mobile application may prompt the user for pet data related to the pet's problem area, as shown in FIGS. 5F-I. For example, the mobile application may display the following prompts: “Is there any redness in your pet's gums?”, “Is there any bleeding from gums or teeth?”, “Any recent change in your pet's appetite?”, “Does your pet have abnormally bad breath?”, and/or “Optional: Provide additional information about condition.”. In response to the prompts, the user may input pet data via the user interface of the mobile application. After inputting the pet data, the user may initiate the submission of the pet data via a widget (e.g., “Submit for Screening”). As shown in FIG. 5J, the mobile application may display an acknowledgment of the receipt of the pet data. The image classifier (e.g., machine-learning model) may analyze the pet image data and the pet data to determine a pet condition. As shown in FIGS. 5K-L, the mobile application may display the pet condition (e.g., “gingivitis”) and a pet condition recommendation (e.g., “A teeth and gum cleaning by your vet is the first course of treatment-prognosis is excellent if treated early.”).

FIGS. 5M-R illustrate exemplary user interfaces for a canine dental check, according to one or more embodiments.

The user interfaces may include asking the user regarding whether the canine is experiencing any symptoms (e.g., blood, oral discomfort). The system may analyze the user responses and/or image data to determine possible diagnoses of the pet.

Additionally, a user interface may output one or more graphics that identify the scanned teeth, teeth with tartar, and/or gum issues near 5 the teeth that were scanned by the system. For example, the graphics may include an image of the teeth, where the graphic may highlight the teeth that were scanned and/or have tartar. A user may interact with the display, where a user may select one tooth via the user interface. In response to the selection, the display may output additional details regarding the particular tooth.

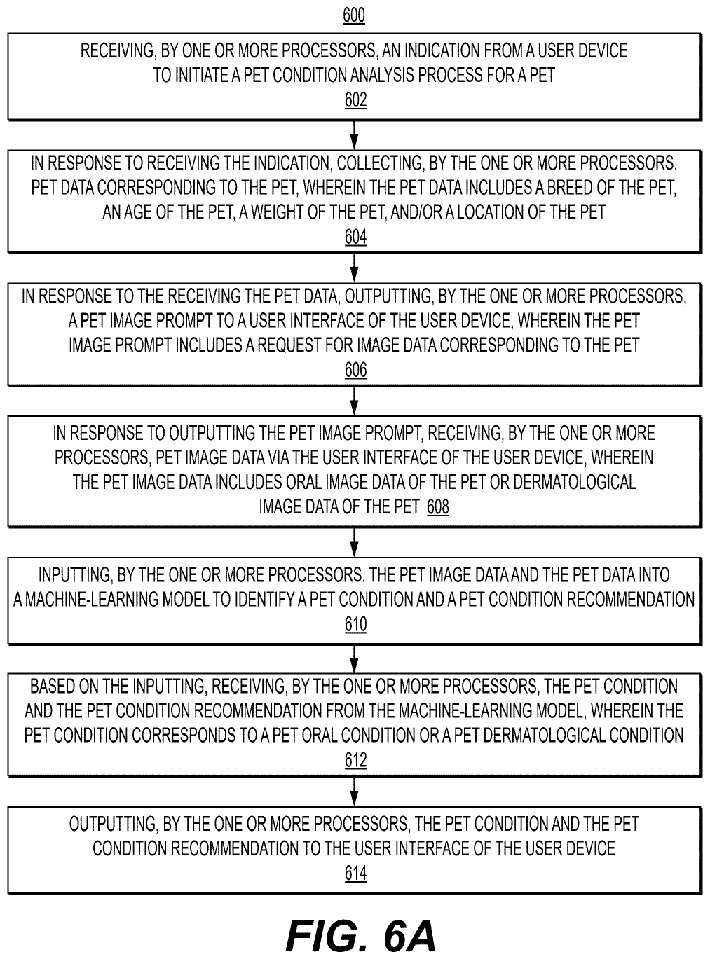

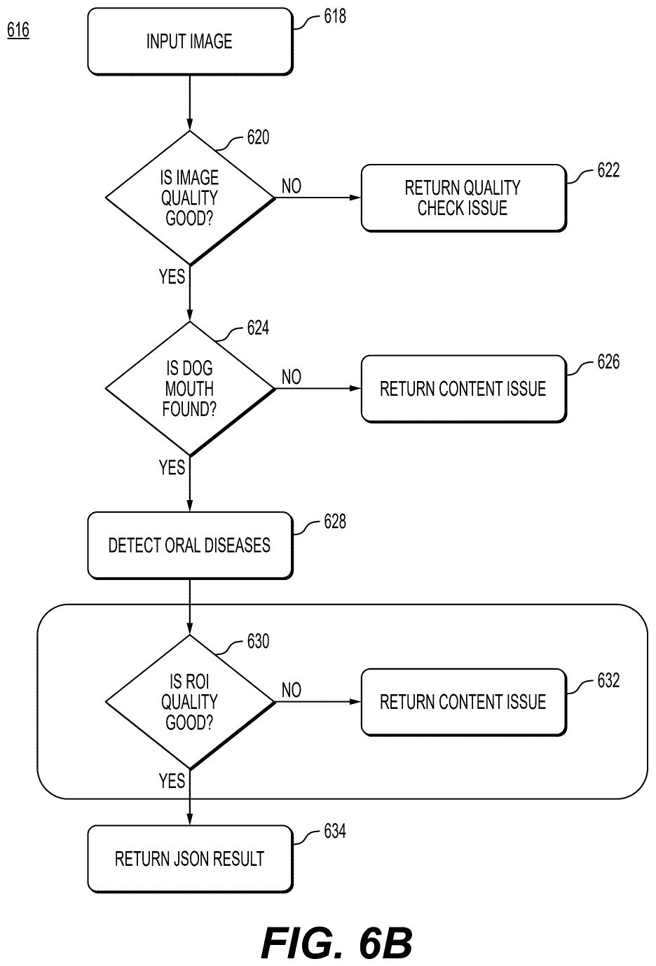

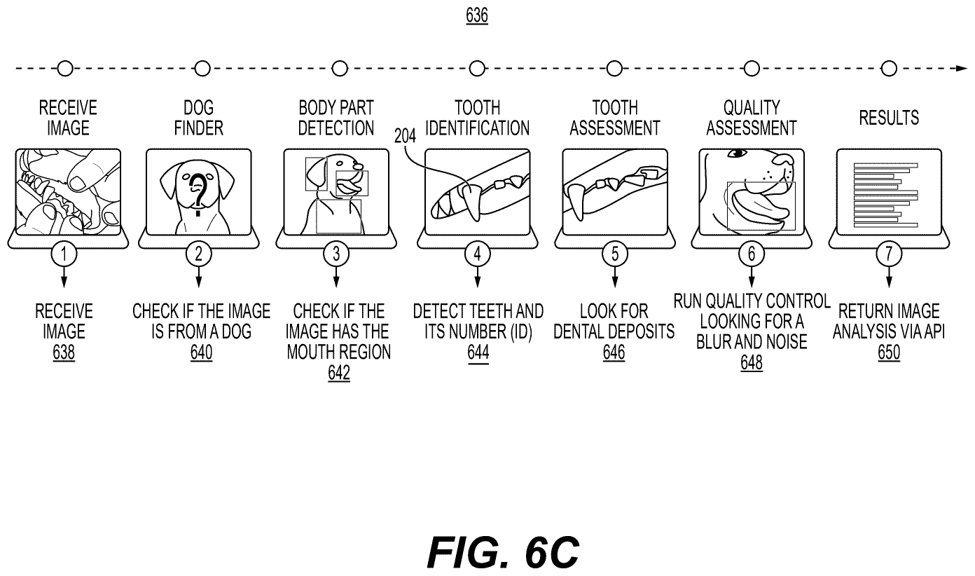

FIG. 6A illustrates a flowchart of an exemplary method 600 of an exemplary embodiment for utilizing an image classifier to identify pet oral conditions and pet dermatological conditions, according to one or more embodiments. Notably, method 600 may be performed by one or more processors of a server that is in communication with one or more user devices and other external system(s) via a network. However, it should be noted that method 600 may be performed by any one or more of the server, one or more user devices, or other external systems. For example, in some embodiments, the method 600 may be performed by a diagnostic system 142, where the diagnostic system 142 may receive pet data from the platform 102, health portal 148, and/or external systems (as described in relation to FIG. 1).

The method may include receiving, by one or more processors, an indication from a user device to initiate a pet condition analysis process for a pet (Step 602). As previously discussed, the user device may execute a mobile application utilizing an image classifier, and display a user interface by which a user may select an option (e.g., a widget) displayed on the user device that may indicate that the user wants to utilize the image classifier. In some embodiments, the user device may initiate the pet condition analysis process by opening a corresponding mobile application.

The method may include, in response to receiving the indication, collecting, by the one or more processors, pet data corresponding to the pet, wherein the pet data includes a breed of the pet, an age of the pet, a weight of the pet, and/or a location of the pet (Step 604). The system may receive the breed of the pet, the age of the pet, and/or the location of the pet (e.g., the city, state, and/or country of the pet) from a web platform (e.g., platform 102). Upon collecting the pet data, the system may store the pet data in a database (e.g., database(s) 104 and/or cloud storage 114). For example, the system may store the pet data in a database record (e.g., in database(s) 104 and/or cloud storage 114) that corresponds to the user (e.g., a pet owner profile 120 or a pet profile 118). In some embodiments, the system may receive the pet data from one or more external connected systems (e.g., a wellness system 124, a diagnostic system 142, a homing system 152, a content management system 164, a genetics system 170, and/or a third party services system 182). For example, the system may send one or more queries to the external systems, which may request that the external systems send pet data corresponding to a pet identifier of the pet to the system.

In some embodiments, the collecting may include outputting, by the one or more processors, a pet data prompt to the user interface of the user device for the pet data. The pet data prompt may correspond to one or more questions for additional information related to the pet. For example, the pet data prompt may be displayed as text on the user interface. Exemplary pet data prompts may be related to the location of the skin issue on the pet (e.g., back, paw, or leg), a sub-location of where the skin issue is located on the pet (e.g., back, stomach, head, neck, armpit, groin, or tail base), what is the main issue (e.g., oozing, odor, or bleeding), and/or how long there has been an issue (e.g., 1-2 days, 3-7 days, 1-2 weeks, or 3 weeks or more). Additionally, or alternatively, the pet data prompt may be generated based on the collected pet data. For example, if the user inputs the dog breed as a “Chihuahua,” the system may generate a pet data prompt related to the small size of the dog. The collecting may include, in response to the outputting the pet data prompt, receiving, by the one or more processors, the pet data that is responsive to the pet data prompt via the user interface of the user device. The user may input responses (e.g., pet data) to the pet data prompt by typing and/or uploaded a recorded response. The system may store the pet data in one or more databases for future reference and/or analysis by the machine-learning model and/or other systems. In some embodiments, the pet data may be stored for future training of the machine-learning model.

In some embodiments, the collecting may include retrieving, by the one or more processors, the pet data from a database that may store pet profile data (e.g., pet profile 204, pet profile 206, pet profile 208). For example, the pet profile data may have been previously stored in a database in an internal system and/or external system. The system may send a request to the database for pet profile data. The request may include a unique pet identifier (e.g., 228) that may correspond to a particular pet profile. In response to receiving the request with the unique pet identifier, the database may access the pet profile that corresponds to the unique pet identifier, and then send the pet profile to the system. The method may include associating the pet profile data with the pet data for the image classifier process.

The method may include embedding, by the one or more processors, the pet data as metadata of the image data. For example, the embedded pet data may be utilized for additional training of the machine-learning model or statistical analysis. The method may also include storing, by the one or more processors, the image data and the metadata in a database. Other systems and/or machine-learning models may access the image data and the embedded metadata for future training.

The method may include, in response to the receiving the pet data, outputting, by the one or more processors, a pet image prompt to a user interface of the user device, wherein the pet image prompt includes a request for image data corresponding to the pet (Step 606). The user interface may display a pet image prompt that requests the user to upload image data (e.g., one or more photos) of the pet. The pet image prompt may request that the image data focus on a particular area of the pet. The pet image prompt may request that the user capture and upload photos that focus on the area of the pet that is exhibiting symptoms. For example, the pet image prompt may ask the user to upload pictures of the pet's teeth.

In some embodiments, the method may include generating, by the one or more processors, the pet image prompt based on the pet data. For example, the pet image prompt may relate to the area of the condition on the pet's body (e.g., “Please upload pictures of both the pet's ears”), the type of condition (e.g., “Please upload pictures of the pet's teeth”), and the like.

The method may include, in response to outputting the pet image prompt, receiving, by the one or more processors, pet image data via the user interface of the user device, wherein the pet image data includes oral image data of the pet or dermatological image data of the pet (Step 608) (e.g., Step 334). The oral image data may include photos of the pet's teeth. The dermatological image data may include pictures of the pet's skin and/or areas of the pet that are experiencing symptoms. The user may upload images that were previously stored on the user device. Additionally, or alternatively, the user may take a photo and directly upload the photo to the image classifier.

In some embodiments, the method may include outputting, by the one or more processors, a label prompt to the user device, wherein the label prompt corresponds to one or more specific symptoms (e.g., oozing, bleeding, missing teeth) of one or more pet conditions. The label prompt may ask the user to annotate (e.g., label) at least one part of the image data. The annotation may indicate a particular area or symptoms that were captured in the image data. For example, the annotation may include an arrow pointing to a particular area of the pet that was captured in the image data, where a label next to the arrow may state “bleeding.”

In some embodiments, the method may also include, in response to outputting the label prompt, receiving, by the one or more processors, a label corresponding to a location of the image data, wherein the label includes a custom label or at least one of a set of labels. The label prompt may include the image data, where the user interface may be configured to allow the user to input the label. The label may include text and/or images (e.g., an arrow) that indicate the one or more specific symptoms. The custom label may correspond to a label that the user creates and/or positions. For example, the user may create an arrow that is directed towards a missing tooth and then add text that states “missing tooth.” The set of labels may correspond to pre-determined labels that were suggested by, for example, the image classifier. The set of labels may include, for example, “bleeding,” “oozing,” and the like. In some embodiments, the set of labels may correspond to the pet data. For example, if the pet data indicates a possible oral condition, the set of labels may include “broken tooth,” “red gums,” “decay,” and the like. In some embodiments, the set of labels may be output to the user device, where the set of labels are output to the user interface of the user device. The user may select a label via the user interface.

The method may include inputting, by the one or more processors, the pet image data and the pet data into a machine-learning model (e.g., Step 336 and/or Step 338) to identify a pet condition and a pet condition recommendation (Step 610). In some embodiments, the machine-learning model may include a computer vision algorithm that may have been trained based on a plurality of oral condition datasets or a plurality of dermatological condition datasets. In addition to the pet image data and the pet data, the label may also be input into the machine-learning model. The machine-learning model may then analyze the pet image data, the pet data, and/or the label to determine a pet condition and a pet condition recommendation. In some embodiments, the label may have been received from the user, as previously discussed. Additionally, or alternatively, the label may have been received from an external system and/or an external user. For example, the label may have been received from a veterinarian, where the labeled pet image data may have been received from another system (e.g., external services 150) and incorporated into the analysis by the image classifier. The pet condition may correspond to an oral condition and/or a dermatological condition. For example, the pet condition may include at least one of: an allergic dermatitis condition, a flea allergy condition, a dermatitis condition, a mange condition, a yeast infection condition, a hot spot condition, a bacterial infection condition, a ringworm condition, a gingivitis condition, a periodontitis condition, a broken teeth condition, an abscess condition, a dental tartar condition, a malocclusion condition, a gingival recession condition, a plaque condition, a calculus condition, a fractured tooth condition, a furcation exposure condition, a bruised tooth condition, a papilloma virus condition, an oral mass condition, a persistent deciduous tooth condition, an oral cancer condition, and the like. The pet condition recommendation may include at least one of: a treatment option, a medication, a set of home care instructions, or follow-up care instructions. The treatment option recommendation may include a plan of how to treat the particular condition, where the treatment option recommendation may include a treatment by a veterinarian and/or medical professional. The medication recommendation may correspond to one or more medications and/or supplements that the pet should take to address the condition. The set of home care instructions may include steps that the user should follow to address the condition. The follow-up care instructions may correspond to one or more professionals that the user should follow-up with to address the condition.

The machine-learning model may have been previously trained based on training data, as previously described. The training data may include training pet image data, training pet data, training labels, training pet conditions, and/or training pet condition recommendations. The training pet image data may include one or more images of a pet's condition. The training pet data may include pet data that corresponds to the pet in each of the training pet image data. The training labels may include at least one label that corresponds to each of the training pet image data. The training pet conditions may include a pet condition that corresponds to each of the images of the pet's condition. The training pet recommendations may correspond to a recommendation to address each of the training pet conditions. The machine-learning model may receive the training data, and then analyze the training data to learn associations between the training data.

The method may include, based on the inputting, receiving, by the one or more processors, the pet condition and the pet condition recommendation from the machine-learning model, wherein the pet condition corresponds to a pet oral condition or a pet dermatological condition (Step 612) (e.g., Step 340). The machine-learning model may determine the pet condition and the pet condition recommendation based on the pet image data, the pet data, and/or the label. Upon determining the pet condition and the pet condition recommendation, the machine-learning model may output the pet condition and the pet condition recommendation to the system.

For example, one layer of the machine-learning model may analyze the pet image data to determine one or more teeth in the pet's mouth. The layer may then apply a bounding box to each tooth, where the bounding box may estimate the location of a tooth. A second layer of the machine-learning model may then analyze each pixel within the bounding box to determine a mask that precisely corresponds to the pet tooth. A third layer of the machine-learning model may then analyze each mask to classify the tooth as having one or more conditions. For example, the masking process allows for the machine-learning model to isolate the particular tooth, thereby reducing noise, focusing on relevant features, and improving the accuracy of the classification process.

The method may include outputting, by the one or more processors, the pet condition and the pet condition recommendation to the user interface of the user device (Step 614). The user interface may display a representation corresponding to the pet condition and/or the pet condition recommendation. In some embodiments, the user interface may also display references (e.g., links) to external systems that may contain additional resources related to the pet condition recommendation.

The method may include generating, by the one or more processors via the machine-learning model, annotated image data that includes the image data and a corresponding annotation that indicates a feature of the pet condition. The machine-learning model may have been previously trained to identify features of the pet condition in the pet image data, where the features may correspond to symptoms of the particular pet condition. For example, the machine-learning model may identify a missing tooth, a crack in a tooth, a rash, bleeding, and the like. The machine-learning model may have also been previously trained to annotate pet image data to indicate the characteristic. The method may include outputting, by the one or more processors, the annotated image data to the user interface of the user device. For example, the user interface may display the pet image data with the annotations that highlight the features of the pet image data.

In some embodiments, the method may include receiving, by the one or more processors, a confidence level from the machine-learning model, wherein the confidence level corresponds to the pet condition. For example, the machine-learning model may generate a confidence level that may correspond to how confident the machine-learning model was regarding the determined the pet condition, the annotated image data, and/or the pet condition recommendation. The confidence level may be described as a ratio (e.g., 10%), text (“low confidence”), and the like. In some embodiments, the method may include outputting, by the one or more processors, the confidence level to the user interface of the user device. For example, the user interface may display the confidence level.

In some embodiments, the method may include storing, by the one or more processors, the pet image data, the pet data, the pet condition, the pet condition recommendation, the label, the confidence level, and/or the annotated image data in one or more databases. Additionally, the machine-learning model may access the databases to utilize the stored data for training, tuning, and analytics purposes.

In an exemplary embodiment, the method may include building a series of computer vision models (e.g., instance segmentation, classification) for detecting one or more dental conditions (e.g., tartar, gingivitis) through an image analysis of image data (e.g., photograph). Additionally, or alternatively, the computer vision models may classify pets (e.g., a dog classifier) captured in the image data, classify body parts of the pet (e.g., a dog body part classifier) captured in the image data, and/or classify the image data based on the image quality (e.g., an image quality classifier). The system may deploy one or more computer vision models as an application programming interface (API). In some embodiments, the API may have a minimum threshold of one tooth in the image data to generate an output. Additionally, the system may connect to a Software Development Kit (SDK) if there are a higher amount of teeth captured in the image data (e.g., five teeth). The process of utilizing the computer vision models may result in an easy and convenient routine for dental monitoring between veterinary visits of the pet. The process may also generate a greater awareness of the importance of oral health in dogs and may support the early identification of oral health problems of the pet.

For example, the method of building and utilizing the computer vision models may include receiving a plurality of crowd-sourced images (e.g., image data) of one or more canine mouths. One or more users (e.g., trained experts) may have previously labeled the images. The labels may indicate particular ailments, conditions, and/or features of the canine mouths. Upon receiving the crowd-sourced images, the system may store the crowd-sourced images and/or collected pet data in one or more databases. The computer vision models may then access the stored crowd-sourced images and/or the collected pet data for analyzing the images, as well as for training and tuning purposes.

The method may further include performing a hierarchical analysis on the plurality of crowd-sourced images (e.g., pet image data). The hierarchical analysis may include filtering the plurality of crowd-sourced images based on image quality and relevant morphological features. For example, the system (e.g., computer vision models) may select a subset of the crowd-sourced images that have an image quality that meets or surpasses a particular threshold. Additionally, or alternatively, the system may reduce the subset to include crowd-sourced images that include morphological features that are relevant for the analysis (e.g., images that include teeth for a tartar analysis). The hierarchical analysis may also include detecting and/or localizing one or more dental conditions. For example, the computer vision models may analyze the subset of images to detect a dental condition that is reflected in the subset of images. The hierarchical analysis may include first confirming that the subject in an image includes a canine. Upon confirming that the subject is a canine, the hierarchical analysis may further include detecting the presence of a mouth in the image. The hierarchical analysis may further include performing a tooth scan by determining between teeth with and without dental deposits. The hierarchical analysis may further include performing a gum scan by identifying gums with gum inflammation within the image. The hierarchical analysis may further include utilizing an image quality classifier, which may evaluate the quality of the region of interest (e.g., the smallest area encompassing all teeth and gums), within the image. After the hierarchical analysis, the system may output the dental condition to a display of a device.

In some embodiments, filtering the pet image data may include utilizing a low quality image classifier, which may be configured to check the quality of an image sent by a user. Tooth and gum machine-learning models have a better performance and increased accuracy when analyzing images that are sharp and have good lighting. To ensure that the predictions of the machine-learning models are more accurate and to improve the user's experience, output of low-quality images may be discarded or presented with a disclaimer. The low quality image classifier may have been trained with one or more datasets that include low quality classes, such as blur, motion blur, dark, exposure, glare, noise, and/or blur-noise.

Using the low quality image classifier may include selecting a region of interest for each image (e.g., using manually annotated gums). The region of interest may cover the visible inner mouth of the canine, where performing a quality check on this region may provide a better understanding regarding the quality of the region where the machine-learning model may make the majority of the predictions. Performing a quality check on the entire image may generate a high number of false positives and false negatives where the region of interest may be significantly smaller in size than the entire image.

False positive detections in tooth identification typically exhibit high blurriness (low sharpness), low brightness values, and/or an appearance in darker regions of the image. In order to avoid false positives, the method may include selecting a region of the image data (e.g., that corresponds to a detected tooth), and then applying one or more filtering methodologies to the region to confirm the existence of a tooth. Exemplary methods for selecting the region may include cropping the image, selecting masked pixels only (one dimension), selecting a masked region and a black fill, and/or selecting a masked region and a mean fill of the image. Exemplary filtering methodologies may include detecting a blurriness metric (e.g., Laplacian variance) for each detected tooth and/or detecting a brightness metric (e.g., mean pixel intensity) for each detected tooth. The filtering methodologies may include a global thresholding technique, which uses a global mean and standard deviation across all tooth detections to calculate the thresholds (e.g., Threshold=mean−K*standard deviation with K=2). The filtering techniques may also include a global and per image threshold, which combines the global threshold with thresholds computed per image (e.g., Threshold=mean−K*standard deviation with K=2). The filtering techniques may further include a global and per detection threshold (KNN), which combines the global threshold with thresholds computed using 5 nearest neighbor detections based on a spatial proximity (e.g., Threshold=mean−K*standard deviation with K=2). Images with blurriness or brightness below the threshold (e.g., global threshold, global and per image threshold, or global and per detection threshold) may be removed.

FIG. 6B illustrates a flowchart of an exemplary method 616 for utilizing an image classifier to determine the image quality of an uploaded image, in relation to method 616, according to one or more embodiments. Notably, method 616 may be performed by one or more processors of a server that is in communication with one or more user devices and other external system(s) via a network. However, it should be noted that method 616 may be performed by any one or more of the server, one or more user devices, or other external systems. For example, in some embodiments, the method 616 may be performed by a diagnostic system 142.

The method may include receiving the input image, where the input image may correspond to image data of a pets mouth (Step 618). The method may include determining whether the image quality is acceptable (e.g., good) (Step 620). For example, the system may determine whether the image quality meets a threshold, where the threshold may correspond to a particular visibility and/or clarity of the image. If the image quality does not meet a threshold, the system may return a notification that indicates a quality check issue (Step 622). If the image quality does meet the threshold, the system may determine whether the image data includes the mouth of a dog (Step 624). The determining may include utilizing one or more machine-learning models. For example, a first machine-learning model may detect the presence of any dog part, where a second machine-learning model may classify/detect a specific dog part (e.g., a dog's mouth). If the image data does not include the mouth of a dog, the system may return a notification that indicates a content issue (Step 626). If the image data does include a dog's mouth, the method may continue with detecting whether the pet has one or more oral diseases, as described above (e.g., in steps 610 and 612) (Step 628). Upon detecting the one or more oral diseases, the system may determine whether the region of interest (“ROI”) quality is acceptable (e.g., good) (Step 630). For example, an acceptable ROI may include a dilated bounding box that covers all of the detections (e.g., gums and teeth) from the machine-learning model(s). If the ROI is not acceptable, the system may return a content issue notification (Step 632). If the ROI is acceptable, the system may return a result (e.g., in a JSON format) that includes details regarding the detected oral disease (Step 634).

FIG. 6C illustrates a flowchart of an exemplary process 636 for performing an oral image analysis of a dog, according to one or more embodiments. Notably, process 636 may be performed by one or more processors of a server that is in communication with one or more user devices and other external system(s) via a network. However, it should be noted that process 636 may be performed by any one or more of the server, one or more user devices, or other external systems. For example, in some embodiments, the process 636 may be performed by a diagnostic system 142.

The process may include receiving an image of a dog's mouth (Step 638). As previously described, a user may take a photo of the dog's mouth, and then upload the photo to the system for performing the oral image analysis. Additionally, as previously described, the system may utilize a custom low quality image classifier trained on images captured by end users to perform a quality check of the image data. The classifier can be a model like RandomForest, XGBoost, Support Vector Machine trained on features extracted from images.

The process may include analyzing the image to confirm that the image is of a dog (Step 640). For example, a machine-learning model may analyze the image to determine whether the image include attributes of a dog (e.g., zoomed-in dog).

Upon confirming that the image is of a dog (e.g., zoomed-in dog), the process may include determining if the image includes a mouth region of the dog (Step 642). For example, a machine-learning model may be used to classify if the image (e.g., a zoomed-in image) is a mouth or any other body part of a dog. Upon detecting a mouth, the process may include detecting individual teeth and a corresponding tooth identification number (Step 644). Detecting individual teeth may include using a machine-learning model to apply a mask to each tooth.

After detecting the individual teeth, the process may include analyzing each tooth and/or the surrounding gum area (Step 646). For example, a machine-learning model may analyze each mask to determine whether the tooth has one or more conditions (e.g., dental deposits). In some embodiments, the machine-learning model may determine that each tooth and/or gum area is healthy or has at least one condition (as described above).

After assessing the individual teeth, the process may include performing a quality assessment to look for blurring and/or noise in the image (Step 648). In some embodiments, if the system determines that the image quality does not meet or exceed a quality threshold, the system may reject the image and/or output a notification to the user that the image does not meet the quality standards.

After performing the quality assessment, the process may include outputting the results to the user device (Step 650). Outputting the results may include outputting a notification that indicates whether the image met or surpassed the image quality threshold. Additionally, outputting the results may include outputting a graphic that includes a representation of the dog's mouth and indicates potential conditions for each tooth.

Although FIGS. 6A, 6B, and 6C show example blocks of exemplary methods 600, 616, and 636, in some implementations, the exemplary methods 600, 616, and 636 may include additional blocks, fewer blocks, different blocks, or differently arranged blocks than those depicted in FIGS. 6A, 6B, and 6C. Additionally, or alternatively, two or more of the blocks of the exemplary methods 600, 616, and 636 may be performed in parallel.

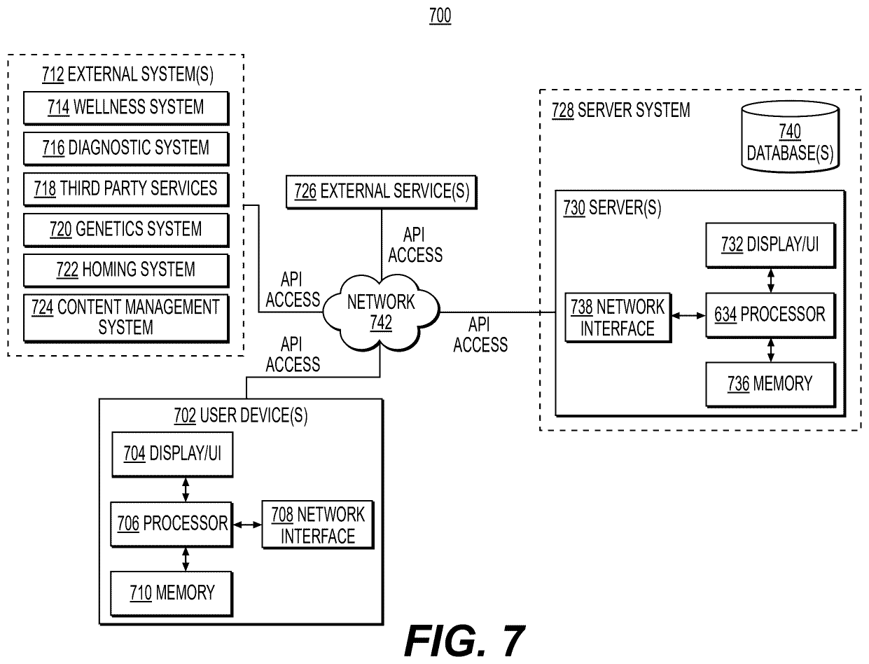

FIG. 7 depicts an exemplary environment 700 that may be utilized with the techniques presented herein. One or more user device(s) 705, one or more external system(s) 726, and one or more server system(s) 728 may communicate across a network 742. As will be discussed in further detail below, one or more server system(s) 728 may communicate with one or more of the other components of the environment 700 across network 742. The one or more user device(s) 702 may be associated with a user, e.g., a user associated with at least one pet.

In some embodiments, the components of the environment 700 are associated with a common entity, e.g., a veterinarian, clinic, animal specialist, research center, pharmaceutical company, or the like. In some embodiments, one or more of the components of the environment may be associated with a different entity than another. The systems and devices of the environment 700 may communicate in any arrangement. As will be discussed herein, systems and/or devices of the environment 700 may communicate in order to receive, send, and/or store data.

The user device 702 may be configured to enable the user to access and/or interact with other systems in the environment 700. For example, the user device 702 may be a computer system such as, for example, a desktop computer, a mobile device, a tablet, etc. In some embodiments, the user device 702 may include one or more electronic application(s), e.g., a program, plugin, browser extension, etc., installed on a memory of the user device 702. In some embodiments, the user device 702 may include a smart collar 730, a smart bed 732, a smart feeder 734, a smart litter box 736, a smart camera 738, and/or the other sensors for collecting a digital image of a pet's (e.g., canine's) life 740

The user device 702 may include a display/user interface (UI) 704, a processor 706, a memory 710, and/or a network interface 708. The user device 702 may execute, by the processor 706, an operating system (O/S) and at least one electronic application (each stored in memory 710). The electronic application may be a desktop program, a browser program, a web client, or a mobile application program (which may also be a browser program in a mobile O/S), an applicant specific program, system control software, system monitoring software, software development tools, or the like. For example, environment 700 may extend information on a web client that may be accessed through a web browser. In some embodiments, the electronic application(s) may be associated with one or more of the other components in the environment 700. The application may manage the memory 710, such as a database, to transmit streaming data to network 742. The display/UI 704 may be a touch screen or a display with other input systems (e.g., mouse, keyboard, etc.) so that the user(s) may interact with the application and/or the O/S. The network interface 708 may be a TCP/IP network interface for, e.g., Ethernet or wireless communications with the network 742. The processor 706, while executing the application, may generate data and/or receive user inputs from the display/UI 704 and/or receive/transmit messages to the server system 728, and may further perform one or more operations prior to providing an output to the network 742.

External system(s) 712 may be, for example, one or more systems that collect, manage, and/or store data corresponding to one or more pets and/or one or more pet owners. The one or more external systems may include at least one of a wellness system 714, a diagnostic system 716, a third party services system 718, a genetics system 720, a homing system 722, and/or a content management system 724. External system(s) 712 may be in communication with other device(s) or system(s) in the environment 700 over the one or more networks 742. For example, external system(s) 712 may communicate with the server system 728 via API (application programming interface) access over the one or more networks 742, and also communicate with the user device(s) 702 via web browser access over the one or more networks 742.

External service(s) 726 may be, for example, one or more third party and/or auxiliary systems that integrate and/or communicate with the server system 728 in performing various document information extraction tasks. External service(s) 726 may be in communication with other device(s) or system(s) in the environment 700 over the one or more networks 742. For example, external service(s) 726 may communicate with the server system 728 via API access over the one or more networks 742, and also communicate with the user device(s) 702 via web browser access over the one or more networks 742.

In various embodiments, the network 742 may be a wide area network (“WAN”), a local area network (“LAN”), a personal area network (“PAN”), or the like. In some embodiments, network 742 may include the Internet, and information and data provided between various systems occurs online. “Online” may mean connecting to or accessing source data or information from a location remote from other devices or networks coupled to the Internet. Alternatively, “online” may refer to connecting or accessing a network (wired or wireless) via a mobile communications network or device. The Internet is a worldwide system of computer networks-a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

The server system 728 may include an electronic data system, e.g., a computer-readable memory such as a hard drive, flash drive, disk, etc. In some embodiments, the server system 728 includes and/or interacts with an application programming interface for exchanging data to other systems, e.g., one or more of the other components of the environment.

The server system 728 may include a database(s) 740 and server(s) 730. The server system 728 may be a computer, system of computers (e.g., rack server(s)), and/or or a cloud service computer system. The server system may store or have access to database(s) 740 (e.g., hosted on a third party server or in memory 738). The server(s) may include a display/UI 732, a processor 734, a memory 736, and/or a network interface 738. The display/UI 732 may be a touch screen or a display with other input systems (e.g., mouse, keyboard, etc.) for an operator of the server(s) 730 to control the functions of the server(s) 730. The server system 728 may execute, by the processor 734, an operating system (O/S) and at least one instance of a servlet program (each stored in memory 736).

Although depicted as separate components in FIG. 7, it should be understood that a component or portion of a component in the environment 700 may, in some embodiments, be integrated with or incorporated into one or more other components. For example, a portion of the display 732 may be integrated into the user device 702 or the like. In some embodiments, operations or aspects of one or more of the components discussed above may be distributed amongst one or more other components. Any suitable arrangement and/or integration of the various systems and devices of the environment 700 may be used.

In general, any process or operation discussed in this disclosure that is understood to be computer-implementable, such as the processes illustrated in FIGS. 1-6, may be performed by one or more processors of a computer system, such any of the systems or devices in the environment 700 of FIG. 7, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices in FIG. 7. One or more processors of a computer system may be included in a single computing device or distributed among a plurality of computing devices. A memory of the computer system may include the respective memory of each computing device of the plurality of computing devices.

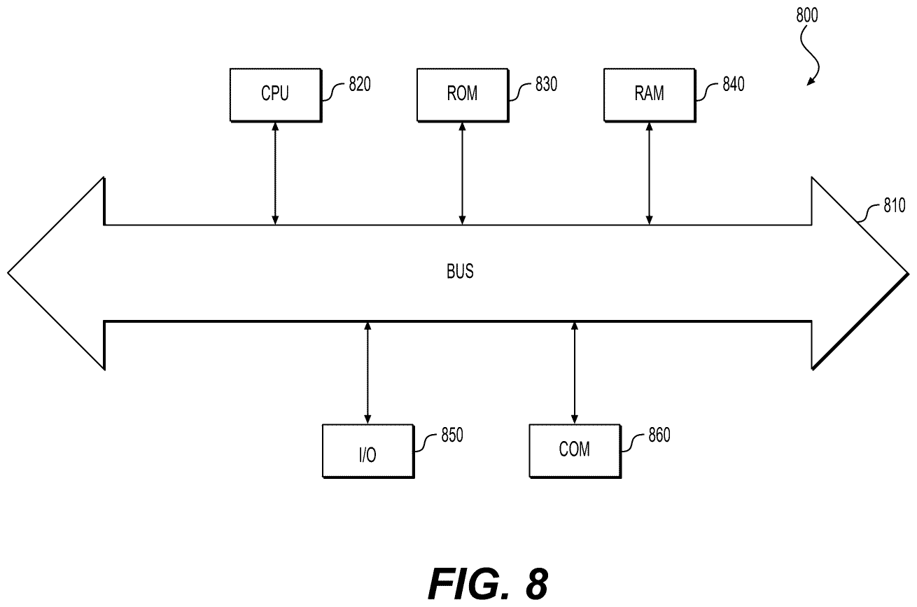

FIG. 8 is a simplified functional block diagram of a computer 800 that may be configured as a device for executing the environments and/or the methods of FIGS. 1-6, according to exemplary embodiments of the present disclosure. For example, device 800 may include a central processing unit (CPU) 820. CPU 820 may be any type of processor device including, for example, any type of special purpose or a general-purpose microprocessor device. As will be appreciated by persons skilled in the relevant art, CPU 820 also may be a single processor in a multi-core/multiprocessor system, such system operating alone, or in a cluster of computing devices operating in a cluster or server farm. CPU 820 may be connected to a data communication infrastructure 810, for example, a bus, message queue, network, or multi-core message-passing scheme.

Device 800 also may include a main memory 840, for example, random access memory (RAM), and also may include a secondary memory 830. Secondary memory 830, e.g., a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage unit may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 830 may include other similar means for allowing computer programs or other instructions to be loaded into device 800. Examples of such means may include a program cartridge and cartridge interface (such as that found in video game devices), a removable memory chip (such as an EPROM, or PROM) and associated socket, and other removable storage units and interfaces, which allow software and data to be transferred from a removable storage unit to device 800.

Device 800 also may include a communications interface (“COM”) 860. Communications interface 860 allows software and data to be transferred between device 800 and external devices. Communications interface 860 may include a modem, a network interface (such as an Ethernet card), a communications port, a PCMCIA slot and card, or the like. Software and data transferred via communications interface 860 may be in the form of signals, which may be electronic, electromagnetic, optical, or other signals capable of being received by communications interface 860. These signals may be provided to communications interface 860 via a communications path of device 800, which may be implemented using, for example, wire or cable, fiber optics, a phone line, a cellular phone link, an RF link or other communications channels.

The hardware elements, operating systems and programming languages of such equipment are conventional in nature, and it is presumed that those skilled in the art are adequately familiar therewith. Device 800 also may include input and output ports 850 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

A computer system, such as a system or device implementing a process or operation in the examples above, may include one or more computing devices. One or more processors of a computer system may be included in a single computing device or distributed among a plurality of computing devices. A memory of the computer system may include the respective memory of each computing device of the plurality of computing devices.

A computer may be configured as a device for executing the exemplary embodiments of the present disclosure. For example, the computer may be configured according to exemplary embodiments of this disclosure. In various embodiments, any of the systems herein may be a computer including, for example, a data communication interface for packet data communication. The computer also may include a central processing unit (“CPU”), in the form of one or more processors, for executing program instructions. The computer may include an internal communication bus, and a storage unit (such as ROM, HDD, SDD, etc.) that may store data on a computer readable medium, although the computer may receive programming and data via network communications. The computer may also have a memory (such as RAM) storing instructions for executing techniques presented herein, although the instructions may be stored temporarily or permanently within other modules of computer (e.g., processor and/or computer readable medium). The computer also may include input and output ports and/or a display to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Furthermore, while some embodiments described herein include some but not other features included in other embodiments, combinations of features of different embodiments are meant to be within the scope of the invention, and form different embodiments, as would be understood by those skilled in the art.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for using an image classifier to identify pet oral conditions and pet dermatological conditions, the computer-implemented method comprising:
receiving, by one or more processors, an indication from a user device to initiate a pet condition analysis process for a pet;
in response to receiving the indication, collecting, by the one or more processors, pet data corresponding to the pet, wherein the pet data includes a breed of the pet, an age of the pet, a weight of the pet, and/or a location of the pet;
in response to the receiving the pet data, outputting, by the one or more processors, a pet image prompt to a user interface of the user device, wherein the pet image prompt includes a request for image data corresponding to the pet;
in response to outputting the pet image prompt, receiving, by the one or more processors, pet image data via the user interface of the user device, wherein the pet image data includes oral image data of the pet or dermatological image data of the pet;
inputting, by the one or more processors, the pet image data and the pet data into a machine-learning model to identify a pet condition and a pet condition recommendation;
based on the inputting, receiving, by the one or more processors, the pet condition and the pet condition recommendation from the machine-learning model, wherein the pet condition corresponds to a pet oral condition or a pet dermatological condition; and
outputting, by the one or more processors, the pet condition and the pet condition recommendation to the user interface of the user device.

2. The computer-implemented method of claim 1, the computer-implemented method further comprising:
outputting, by the one or more processors, a label prompt to the user device, wherein the label prompt corresponds to one or more specific symptoms of one or more pet conditions; and
in response to outputting the label prompt, receiving, by the one or more processors, a label corresponding to a location of the image data, wherein the label includes a custom label or at least one of a set of labels.

3. The computer-implemented method of claim 2, wherein the set of labels are output to the user device, and wherein the set of labels correspond to the pet data.

4. The computer-implemented method of claim 1, wherein the machine-learning model includes a computer vision algorithm that was trained based on a plurality of oral condition datasets or a plurality of dermatological condition datasets.

5. The computer-implemented method of claim 1, the computer-implemented method further comprising:
receiving, by the one or more processors, a confidence level from the machine-learning model, wherein the confidence level corresponds to the pet condition; and
outputting, by the one or more processors, the confidence level to the user interface of the user device.

6. The computer-implemented method of claim 1, the computer-implemented method further comprising:
generating, by the one or more processors via the machine-learning model, annotated image data that includes the image data and a corresponding annotation that indicates a feature of the pet condition; and
outputting, by the one or more processors, the annotated image data to the user interface of the user device.

7. The computer-implemented method of claim 1, the computer-implemented method further comprising:
embedding, by the one or more processors, the pet data as metadata of the image data; and
storing, by the one or more processors, the image data and the metadata in a database.

8. The computer-implemented method of claim 1, wherein collecting the pet data corresponding to the pet further comprises:
outputting, by the one or more processors, a pet data prompt to the user interface of the user device for the pet data; and
in response to the outputting the pet data prompt, receiving, by the one or more processors, the pet data that is responsive to the pet data prompt via the user interface of the user device.

9. The computer-implemented method of claim 1, wherein collecting the pet data further comprises:
retrieving, by the one or more processors, the pet data from a databases that stores pet profile data.

10. The computer-implemented method of claim 1, the computer-implemented method further comprising:
generating, by the one or more processors, the pet image prompt based on the pet data.

11. The computer-implemented method of claim 1, wherein the pet condition includes at least one of: an allergic dermatitis condition, a flea allergy condition, a dermatitis condition, a mange condition, a yeast infection condition, a hot spot condition, a bacterial infection condition, a ringworm condition, a gingivitis condition, a periodontitis condition, a broken teeth condition, an abscess condition, a dental tartar condition, a malocclusion condition, a gingival recession condition, a plaque condition, a calculus condition, a fractured tooth condition, a furcation exposure condition, a bruised tooth condition, a papilloma virus condition, an oral mass condition, a persistent deciduous tooth condition, and/or an oral cancer condition.

12. The computer-implemented method of claim 1, wherein the pet condition recommendation includes at least one of: a treatment option, a medication, a set of home care instructions, or follow-up care instructions.

13. A computer system for using an image classifier to identify pet oral conditions and pet dermatological conditions, the computer system comprising:
at least one memory storing instructions; and
at least one processor configured to execute the instructions to perform operations comprising:
receiving an indication from a user device to initiate a pet condition analysis process for a pet;
in response to receiving the indication, collecting pet data corresponding to the pet, wherein the pet data includes a breed of the pet, an age of the pet, a weight of the pet, and/or a location of the pet;
in response to the receiving the pet data, outputting a pet image prompt to a user interface of the user device, wherein the pet image prompt includes a request for image data corresponding to the pet;
in response to outputting the pet image prompt, receiving pet image data via the user interface of the user device, wherein the pet image data includes oral image data of the pet or dermatological image data of the pet;
inputting the pet image data and the pet data into a machine-learning model to identify a pet condition and a pet condition recommendation;
based on the inputting, receiving the pet condition and the pet condition recommendation from the machine-learning model, wherein the pet condition corresponds to a pet oral condition or a pet dermatological condition; and
outputting the pet condition and the pet condition recommendation to the user interface of the user device.

14. The computer system of claim 13, the operations further comprising:
outputting a label prompt to the user device, wherein the label prompt corresponds to one or more specific symptoms of one or more pet conditions; and
in response to outputting the label prompt a label corresponding to a location of the image data, wherein the label includes a custom label or at least one of a set of labels.

15. The computer system of claim 14, wherein the set of labels are output to the user device, and wherein the set of labels correspond to the pet data.

16. The computer system of claim 14, the operations further comprising:
receiving a confidence level from the machine-learning model, wherein the confidence level corresponds to the pet condition; and
outputting the confidence level to the user interface of the user device.

17. The computer system of claim 14, the operations further comprising:
generating, via the machine-learning model, annotated image data that includes the image data and a corresponding annotation that indicates a feature of the pet condition; and
outputting the annotated image data to the user interface of the user device.

18. A non-transitory computer-readable medium storing instructions that, when executed by at least one processor, cause the at least one processor to perform operations for using an image classifier to identify pet oral conditions and pet dermatological conditions, the operations comprising:
receiving an indication from a user device to initiate a pet condition analysis process for a pet;
in response to receiving the indication, collecting pet data corresponding to the pet, wherein the pet data includes a breed of the pet, an age of the pet, a weight of the pet, and/or a location of the pet;
in response to the receiving the pet data, outputting a pet image prompt to a user interface of the user device, wherein the pet image prompt includes a request for image data corresponding to the pet;
in response to outputting the pet image prompt, receiving pet image data via the user interface of the user device, wherein the pet image data includes oral image data of the pet or dermatological image data of the pet;
inputting the pet image data and the pet data into a machine-learning model to identify a pet condition and a pet condition recommendation;
based on the inputting, receiving the pet condition and the pet condition recommendation from the machine-learning model, wherein the pet condition corresponds to a pet oral condition or a pet dermatological condition; and
outputting the pet condition and the pet condition recommendation to the user interface of the user device.

19. The non-transitory computer-readable medium of claim 18, the operations further comprising:
embedding the pet data as metadata of the image data; and
storing the image data and the metadata in a database.

20. The non-transitory computer-readable medium of claim 18, wherein collecting the pet data corresponding to the pet further comprises:
outputting a pet data prompt to the user interface of the user device for the pet data; and
in response to the outputting the pet data prompt, receiving the pet data that is responsive to the pet data prompt via the user interface of the user device.
